(19) United States
(12) Patent Application Publication
(10) Pub. No.: US 2025/0191676 A1
(43) Pub. Date: Jun. 12, 2025

(54) COMPUTATIONAL-BASED METHODS FOR IMPROVING PROTEIN PURIFICATION

(71) Applicant: Genentech, Inc., South San Francisco, CA (US)

(72) Inventors: Andrew James MAIER, Oakland, CA (US); Sean Mackenzie Burgess, San Francisco, CA (US); Minjeong Cha, Foster City, CA (US)

(21) Appl. No.: 19/053,054

(22) Filed: Feb. 13, 2025

Related U.S. Application Data
(63) Continuation of application No. PCT/US2023/072176, filed on Aug. 14, 2023.
(60) Provisional application No. 63/398,168, filed on Aug. 15, 2022.

(51) Int. Cl. G16B 15/30; G06N 20/10; G16B 5/00; G16B 40/20

(52) U.S. Cl. CPC G16B 15/30; G06N 20/10; G16B 5/00; G16B 40/20

(57) ABSTRACT

A method implemented by one or more computer devices includes accessing a molecular descriptor matrix representing a set of amino acid sequences corresponding to proteins, and refining a set of hyper-parameters associated with a machine learning model trained to generate a prediction of a molecular binding property of the proteins. Refining the set of hyper-parameters comprises iteratively executing a process until a desired precision is reached, including: reducing the molecular descriptor matrix by selecting one representative feature vector for each of a plurality of feature vector clusters, determining most-predictive feature vectors of the selected representative feature vectors based on a correlation, calculating cross-validation losses based on the most-predictive feature vectors and predetermined batch binding data, and updating the set of hyper-parameters based on the cross-validation losses. The method further includes generating the prediction of the molecular binding property of the proteins.

### CROSS-REFERENCE TO RELATED APPLICATION

This application is a continuation of International Application No. PCT/US2023/072176, filed on Aug. 14, 2023, which claims priority to U.S. Provisional Application No. 63/398,168, entitled “COMPUTATIONAL-BASED METHODS FOR IMPROVING PROTEIN PURIFICATION,” which was filed on Aug. 15, 2022, and the disclosure of which is hereby incorporated by reference in its entirety.

### TECHNICAL FIELD

This application relates generally to protein purification, and, more particularly, to computational-based methods for improving protein purification.

### BACKGROUND

Cell cultures utilizing engineered mammalian or bacterial cell lines can be used to produce a target protein of interest by, for example, insertion of a recombinant plasmid containing the gene for the target protein. Because the cell lines themselves are living organisms, the cell lines produce other proteins than the target protein and may require a complex growth medium including, for example, various sugars, amino acids, and growth factors. It is often desired, if not required, to obtain a high-purity composition of the target protein, especially when the target protein is going to be used as a therapeutic active agent, such as when the target protein is a therapeutic antibody. Thus, the produced target protein needs to be purified from these other components in the cell culture, which may involve a complex sequence of processes each involving many variables, such as chromatography stationary phases, mobile phases, salt concentrations, pHs, and other operating conditions, such as temperature.

For example, a sequence of protein purification processes can include: (a) obtaining a cell culture sample containing the target protein; (b) one or more capture steps, such as an affinity capture step using, for example, protein A; (c) one or more conditioning steps; (d) one or more depth filtration steps; (e) one or more ion exchange chromatography steps, such as cation exchange or anion exchange chromatography, or a mixed mode thereof optionally including with hydrophobic interaction chromatography; (f) one or more hydrophobic interaction chromatography steps, or a mixed mode thereof; (g) a virus filtration step; and (h) one or more ultra-filtration steps. Each of such column chromatography techniques may include various conditions utilized to purify the target protein. Specifically, purification techniques include many variables critical to efficiently producing a high-purity composition of the target protein—in addition to considerations regarding the target protein itself, one must consider, for example, the chromatography stationary phase, the mobile phases, salt concentrations, pHs, and other operating conditions, such as temperature.

Currently, purification techniques and such operating conditions are determined experimentally in the laboratory. Thus, determining how to purify a target protein may be very cumbersome and time-consuming. Additionally, relying solely on the foregoing experimental purification techniques to separate and isolate target proteins, useful feedback on which candidate proteins that may be difficult to purify may not be ascertainable without arduous experimentation. This may thus lead to costly inefficiencies in the development and manufacturing processes of therapeutic antibodies or other similar immunotherapies. Accordingly, it may be useful to provide techniques to optimize and streamline the process of protein purification for identifying target proteins (e.g., antibodies) and accelerating the selection process of therapeutic antibody candidates.

### SUMMARY

Embodiments of the present disclosure are directed toward one or more computing devices, methods, and non-transitory computer-readable media that may utilize a machine learning model iteratively trained to generate a prediction of a molecular binding property of one or more target proteins as part of a streamlined process of protein purification for identifying target proteins (e.g., antibodies) and accelerating the selection process of therapeutic antibody candidates or other immunotherapy candidates by way of early identification of the most promising therapeutic antibody candidates. This streamlined process of identifying target proteins (e.g., antibodies) in-silico, for example, may facilitate and accelerate the downstream development and manufacturing of one or more therapeutic monoclonal antibodies (mAbs), bispecific antibodies (bsAbs), trispecific antibodies (tsAbs), or other similar immunotherapies that may be utilized to treat various patient diseases. In some embodiments, the machine learning model comprises an ensemble machine learning model comprising a plurality of models.

For example, once trained, the machine learning model (e.g., “boosting” ensemble-learning model) may be utilized to generate a prediction of a molecular binding property (e.g., a prediction of a percent protein bound at one or more specific pH values and specific salt concentrations and/or specific salt species and chromatographic resin) of one or more proteins by utilizing optimized hyper-parameters (e.g., general parameters, booster parameters, learning-task parameters) and learnable parameters (e.g., regression model weights, decision variables) learned during the training of the machine learning model and a selected k-best matrix of feature vectors of a molecular descriptor matrix representing a set of amino acid sequences corresponding to one or more proteins of interest.

Specifically, in accordance with the presently-disclosed embodiments, once trained, the machine learning model may utilize the optimized hyper-parameters (e.g., general parameters, booster parameters, learning-task parameters) and learnable parameters (e.g., regression model weights, decision variables) learned during training to predict a percent protein bound (e.g., a percentage of a set of proteins predicted to bind to a ligand within a solution for a given pH value and salt concentration) for one or more target proteins based only on, as input, the selected k-best matrix of feature vectors of a molecular descriptor matrix representing a set of amino acid sequences corresponding to one or more proteins of interest and one or more sets of pH values and salt concentrations associated with the binding properties of the one or more proteins of interest.

In this way, by providing a computational-model-based prediction of the percent protein bound for one or more proteins of interest, the molecular binding property and elution property of the one or more proteins of interest may be determined without considerable upstream experimentation. That is, desirable proteins of the one or more proteins of interest may be identified and distinguished from undesirable proteins of the one or more proteins of interest in-silico, and those desirable proteins identified in-silico may be further utilized to facilitate and accelerate the downstream development and manufacturing of one or more therapeutic mAbs, bsAbs, tsAbs, or other similar immunotherapies that may be utilized to treat various patient diseases (e.g., by reducing upstream experimental duration and experimentation inefficiency and providing in-silico feedback on which candidate proteins may be difficult to purify, and, by extension, ultimately difficult to manufacture).

For example, during training of the machine learning model (e.g., “boosting” ensemble-learning model), the hyper-parameters (e.g., general parameters, booster parameters, learning-task parameters) and learnable parameters (e.g., regression model weights, decision variables) may be refined and optimized iteratively. The iterations may include 1) reducing a molecular descriptor matrix representing the set of amino acid sequences by clustering similar feature vectors of the molecular descriptor matrix based on a distance metric. For example, the distance metric may be calculated based on a Pearson's correlation, mutual information, or maximum information coefficient (MIC), or other distance metrics. Feature vectors other than the one closest to the cluster centroid may be discarded to generate a reduced molecular descriptor matrix. The iterations may next include determining the k-best most-predictive feature vectors of the reduced molecular descriptor matrix based on a k-best process and a maximum information coefficient (MIC) for determining a correlation between the feature vectors of the reduced molecular descriptor matrix and an experimentally-determined percent protein bound and/or first principal component (PC) value for one or more specific pH values and salt concentrations. The iterations may next include calculating an n-number of cross-validation losses based on the k-best most-predictive feature vectors and the experimentally-determined percent protein bound and/or the first PC value. Finally, the iterations may include updating the hyper-parameters (e.g., general parameters, booster parameters, learning-task parameters) based on the n-number of cross-validation losses.

In certain embodiments, reducing the molecular descriptor matrix, which may include a large set of amino acid sequence-based descriptors, by way of the foregoing feature dimensionality reduction and feature selection techniques may ensure that the regression model successfully converges to an accurately trained regression model as opposed to suffering overfitting due to superfluous or noisy descriptors. Further, it should be appreciated that, in some embodiments, alternative to utilizing the MIC as part of the determination of the correlation between the feature vectors of the reduced molecular descriptor matrix and the experimentally-determined percent protein bound and/or the first PC value, a distance correlation, mutual information, or other similar nonlinear correlation metric, or a linear correlation metric (e.g., Pearson's correlation), may be utilized.

In certain embodiments, one or more computing devices, methods, and non-transitory computer-readable media may access a molecular descriptor matrix representing a set of amino acid sequences corresponding to one or more proteins. In some embodiments, the molecular descriptor matrix may be generated by a first machine learning model (e.g., a matrix generation machine learning model) distinct from a machine learning model (e.g., an ensemble-learning model). In some embodiments, the first machine learning model was trained to generate the molecular descriptor matrix based on the set of amino acid sequences. For example, in some embodiments, the first machine learning model may include a neural network trained to generate the M×N descriptor matrix representing the set of amino acid sequences, in which N includes a number of the set of amino acid sequences and M includes a number of nodes in an output layer of the neural network.

In certain embodiments, the one or more computing devices may then refine a set of hyper-parameters associated with a machine learning model trained to generate a prediction of a molecular binding property of the one or more proteins. In some embodiments, the machine learning model may include one or more of a gradient boosting model, an adaptive boosting (AdaBoost) model, an extreme gradient boosting (XGBoost) model, a light gradient boosted machine (LightGBM) model, or a categorical boosting (CatBoost) model.

In some embodiments, the prediction of the molecular binding property of the one or more proteins may be generated by a computational model-based column process. In some embodiments, the computational model-based chromatography process may include one or more of a computational model-based affinity chromatography process, an ion exchange chromatography (IEC) process, a hydrophobic interaction chromatography (HIC) process, or a mixed-mode chromatography (MMC) process.

The methods provided herein may be used to predict a molecular binding property of one or more proteins in any chromatography setting, including any series of chromatography techniques or combination of specific operating conditions. Generally, chromatography techniques involve a stationary phase and a mobile phase. The stationary phase may include moieties designed to interact with a target protein (such as in a bind and elute mode style of chromatography) or to not interact with the target protein (such as in a flow through style of chromatography). The mobile phase(s) used in a chromatography technique (such as a loading, washing, or elution mobile phase) may have many variables, including a concentration of one or more salts, pH, and solvent gradients. Additionally, chromatography techniques can be performed in various conditions, such as at elevated temperatures.

In some embodiments, the computational model-based chromatography process include an affinity chromatography process. In some embodiments, the affinity chromatography process may include an affinity ligand, such as according to any of a protein A chromatography, a protein G chromatography, a protein A/G chromatography, a protein L chromatography, and a kappa chromatography. In some embodiments, the affinity chromatography process may include an elution mobile phase, such as a mobile phase having a set pH.

In some embodiments, the computational model-based chromatography process may include an ion exchange chromatography process. Ion exchange chromatography allow for separation based on electrostatic interactions (anion and cation) between a ligand of the ion exchange stationary phase and a component of a sample, for example, a target or non-target protein. In some embodiments, the ion exchange chromatography process a cation exchange (CEX) stationary phase. In some embodiments, the ion exchange chromatography may include a strong CEX stationary phase. In some embodiments, the ion exchange chromatography may include a weak CEX stationary phase. In some embodiments, the ion exchange chromatography resin may be functionalized with ligands containing anionic functional group(s) such as a carboxyl group or a sulfonate group.

In some embodiments, the ion exchange chromatography stationary phase may include an anion exchange (AEX) stationary phase. In some embodiments, the ion exchange chromatography may include a strong AEX stationary phase. In some embodiments, the ion exchange chromatography may include a weak AEX stationary phase. In some embodiments, the ion exchange chromatography resin may be functionalized with ligands containing cationic functional group(s) such as a quaternary amine. In some embodiments, the ion exchange chromatography may include a multimodal ion exchange (MMIEX) stationary phase. MMIEX chromatography stationary phases may include both cation exchange and anion exchange components and/or features. In some embodiments, the MMIEX stationary phase may include a multimodal anion/cation exchange (MM-AEX/CEX) stationary phase.

In some embodiments, the ion exchange chromatography may include a ceramic hydroxyapatite chromatography stationary phase. In some embodiments, the ion exchange chromatography stationary phase may be selected from the group consisting of: sulphopropyl (SP) Sepharose® Fast Flow (SPSFF), quaternary ammonium (Q) Sepharose® Fast Flow (QSFF), SP Sepharose® XL (SPXL), Streamline™ SPXL, ABx™ (MM-AEX/CEX medium), Poros™ XS, Poros™ 50HS, diethylaminoethyl (DEAE), dimethylaminoethyl (DMAE), trimethylaminoethyl (TMAE), quaternary aminoethyl (QAE), mercaptoethylpyridine (MEP)-Hypercel™, HiPrep™ Q XL, Q SepharoseR XL, and HiPrep™ SP XL. In some embodiments, the ion exchange chromatography process may include an elution step mobile phase including increased salt concentrations, such as increased relative to binding or washing mobile phases.

In some embodiments, the computational model-based chromatography process may include a mixed mode chromatography process. Mixed mode chromatography processes may include stationary phases that combine charge-based (i.e., ion exchange chromatography features) and hydrophobic-based elements. In some embodiments, the mixed mode chromatography process may include a bind and elute mode of operation. In some embodiments, the mixed mode chromatography process may include a flow-through mode of operation. In some embodiments, the mixed mode chromatography process may include a stationary phase selected from the group consisting of Capto MMC and Capto Adhere.

In some embodiments, the computational model-based chromatography process may include a hydrophobic interaction chromatography (HIC) process. Hydrophobic interaction chromatography processes may include hydrophobic stationary phases. In some embodiments, the mixed mode chromatography process may include a bind and elute mode of operation. In some embodiments, the hydrophobic interaction chromatography process may include a flow-through mode of operation. In some embodiments, the hydrophobic interaction chromatography process may include a stationary phase including a substrate, such as an inert matrix, for example, a cross-linked agarose, sepharose, or resin matrix. In some embodiments, at least a portion of the substrate of a hydrophobic interaction chromatography stationary phase may include a surface modification including the hydrophobic ligand.

In some embodiments, the hydrophobic interaction chromatography ligand is a ligand including between about 1 and 18 carbons. In some embodiments, the hydrophobic interaction chromatography ligand may include 1 or more carbons, such as any of 2 or more carbons, 3 or more carbons, 4 or more carbons, 5 or more carbons, 6 or more carbons, 7 or more carbons, 8 or more carbons, 9 or more carbons, 10 or more carbons, 11 or more carbons, 12 or more carbons, 13 or more carbons, 14 or more carbons, 15 or more carbons, 16 or more carbons, 17 or more carbons, or 18 or more carbons. In some embodiments, the hydrophobic interaction chromatography ligand may include any of 1 carbon, 2 carbons, 3 carbons, 4 carbons, 5 carbons, 6 carbons, 7 carbons, 8 carbons, 9 carbons, 10 carbons, 11 carbons, 12 carbons, 13 carbons, 14 carbons, 15 carbons, 16 carbons, 17 carbons, or 18 carbons. In some embodiments, the hydrophobic ligand is selected from the group consisting of an ether group, a methyl group, an ethyl group, a propyl group, an isopropyl group, a butyl group, a t-butyl group, a hexyl group, an octyl group, a phenyl group, and a polypropylene glycol group.

In some embodiments, the HIC medium is a hydrophobic charge induction chromatography medium. In some embodiments, the hydrophobic interaction chromatography process may include a mobile phase including a high salt condition. For example, a high salt condition may be used to reduce the solvation of the target thereby exposing hydrophobic regions which can then interact with the hydrophobic interaction chromatography stationary phase. In some embodiments, the hydrophobic interaction chromatography process may include a mobile phase including a low salt condition, for example, with no salt or no added salt. In some embodiments, the hydrophobic interaction chromatography stationary phase is selected from the group consisting of Bakerbond WP HI-Propyl™, Phenyl Sepharose® Fast Flow (Phenyl-SFF), Phenyl Sepharose® Fast Flow Hi-sub (Phenyl-SFF HS), Toyopearl® Hexyl-650, Poros™ Benzyl Ultra, and Sartobind® phenyl. In some embodiments, the Toyopearl® Hexyl-650 is Toyopearl® Hexyl-650M. In some embodiments, the Toyopearl® Hexyl-650 is Toyopearl® Hexyl-650C. In some embodiments, the Toyopearl® Hexyl-650 is Toyopearl® Hexyl-650S.

In certain embodiments, the prediction of the molecular binding property of the one or more proteins may include an identification of a target protein of the one or more proteins. In another embodiment, the prediction of the molecular binding property of the one or more proteins may use quantitative structure property relationship (QSPR) or a quantitative structure activity relationship (QSAR) modeling of the one or more proteins. In another embodiment, the prediction of the molecular binding property of the one or more proteins may include a prediction of a molecular binding property for each amino acid sequence of the set of amino acid sequences corresponding to the one or more proteins. In another embodiment, the prediction of the molecular binding property for each amino acid sequence may include a computational model-based isolation of desirable amino acid molecules from undesirable amino acid molecules. In one embodiment, the machine learning model (e.g., an ensemble-learning model) may be further trained to generate a prediction of a molecular elution property of the one or more proteins. In another embodiment, the machine learning model may be further trained to generate a prediction of a flow-through property of the one or more proteins.

In certain embodiments, the one or more computing devices may refine the set of hyper-parameters iteratively by executing a process until a desired precision is reached. For example, in certain embodiments, the one or more computing devices may execute the process by first reducing the molecular descriptor matrix by selecting one representative feature vector for each of a plurality of feature vector clusters. In one embodiment, each of the feature vector clusters includes similar feature vectors. For example, in some embodiments, reducing the molecular descriptor matrix may include performing clustering using a correlation distance metric, for example, calculated based on a Pearson's correlation of feature vectors of the molecular descriptor matrix, to generate the plurality of feature vector clusters. In some cases, the clustering of the sets of descriptors may be based on the correlation distance between the descriptors, which may be calculated from the Pearson's correlation (e.g., 1-abs (Pearson's Correlation)). In one embodiment, the selected one representative feature vector for each of the plurality of feature vector clusters may include a centroid feature vector for each of the plurality of feature vector clusters utilized to represent two or more of the similar feature vectors.

In certain embodiments, the one or more computing devices may execute the process by then determining one or more most-predictive feature vectors of the selected representative feature vectors for each of the plurality of feature vector clusters based on a correlation between the selected representative feature vectors and predetermined batch binding data associated with the one or more proteins. For example, in some embodiments, determining the one or more representative feature vectors of the selected representative feature vectors for each of the plurality of feature vector clusters may include selecting a k-best matrix of feature vectors of the selected representative feature vectors for each of the plurality of feature vector clusters. In one embodiment, the k-best matrix of feature vectors of the selected representative feature vectors is determined based on a predetermined k-best process. In certain embodiments, the correlation between the selected representative feature vectors and the predetermined batch binding data is determined based on a Pearson's correlation, mutual information, maximal information coefficient (MIC), or other metric, between the selected representative feature vectors and the predetermined batch binding data. In other embodiments, alternative to utilizing the MIC as part of the determination of the correlation, a distance correlation, mutual information, or other similar nonlinear correlation metric and/or linear correlation metrics may be utilized.

In certain embodiments, the one or more computing devices may execute the process by then calculating one or more cross-validation losses based at least in part on the one or more most-predictive feature vectors and the predetermined batch binding data. For example, in some embodiments, calculating the one or more cross-validation losses further may include evaluating a cross-validation loss function based on the one or more most-predictive feature vectors, the predetermined batch binding data, the set of hyper-parameters, and a set of learnable parameters associated with the machine learning model, and further minimizing the cross-validation loss function by varying the set of learnable parameters while the one or more most-predictive feature vectors, the predetermined batch binding data, and the set of hyper-parameters remain constant.

For example, in some embodiments, minimizing the cross-validation loss function may include optimizing the set of hyper-parameters. For example, in one embodiment, the set of hyper-parameters may include one or more of a set of general parameters, a set of booster parameters, or a set of learning-task parameters. In some embodiments, minimizing the cross-validation loss function may further include minimizing a loss between a prediction of a percent protein bound for the one or more proteins and an experimentally-determined percent protein bound for the one or more proteins. In one embodiment, the predetermined batch binding data may include an experimentally-determined percent protein bound for one or more pH values and salt concentrations associated with the molecular binding property of the one or more proteins. In one embodiment, the set of learnable parameters may include one or more weights or decision variables determined by the machine learning model based at least in part on the one or more most-predictive feature vectors and the predetermined batch binding data.

In some embodiments, calculating the one or more cross-validation losses may include calculating an n number of cross-validation losses, in which n includes an integer from 1-n. In some embodiments, calculating the one or more cross-validation losses may include determining an n number of individual train-test splits based on the one or more most-predictive feature vectors and the predetermined batch binding data, in which n includes an integer from 1-n. In some embodiments, calculating the one or more cross-validation losses may include calculating an n number of cross-validation losses and generating the prediction of the molecular binding property of the one or more proteins based on an averaging of the n number of cross-validation losses.

In certain embodiments, the one or more computing devices may execute the process by then updating the set of hyper-parameters based on the one or more cross-validation losses. For example, the updated set of hyper-parameters may include one or more of an updated set of general parameters, an updated set of booster parameters, or an updated set of learning-task parameters. In some embodiments, subsequent to refining the set of hyper-parameters, the one or more computing devices may output, by the machine learning model, the prediction of the molecular binding property of the one or more proteins based at least in part on the updated set of hyper-parameters.

In certain embodiments, subsequent to refining the set of hyper-parameters, the one or more computing devices may further access a second molecular descriptor matrix representing a second set of amino acid sequences corresponding to one or more second proteins, reduce the second molecular descriptor matrix by selecting one representative feature vector for each of a second plurality of feature vector clusters of the second molecular descriptor matrix, determine one or more second most-predictive feature vectors of the selected representative feature vectors for each feature vector cluster based on a second correlation between the selected representative feature vectors and predetermined batch binding data associated with the one or more second proteins. inputting the one or more second most-predictive feature vectors into the machine learning model trained to generate a prediction of a molecular binding property of the one or more second proteins, and outputting, by the machine learning model, the prediction of the molecular binding property of the one or more second proteins based at least in part on the updated set of hyper-parameters. For example, the prediction of the molecular binding property of the one or more second proteins may include a prediction of a second percent protein bound for the one or more second proteins.

In certain embodiments, the one or more computing devices may further optimize the machine learning model based on a Bayesian model-optimization process. In some embodiments, the one or more computing devices may then utilize Group K-Fold cross-validation to train and evaluate the optimized machine learning model based on the one or more most-predictive feature vectors, the predetermined batch binding data, the set of hyper-parameters, and the set of learnable parameters. In some embodiments, the Group K-Fold cross validation may be stratified in order to ensure that the cross-validation training and evaluation splits include a diverse range of regression target values. In some embodiments, the stratification might be accomplished using labels generated by binning the regression target values into a number of quantiles.

In certain embodiments, one or more computing devices, methods, and non-transitory computer-readable media may access a molecular descriptor matrix representing a set of amino acid sequences corresponding to one or more proteins; and obtain, by a machine learning model, a prediction of a molecular binding property of the one or more proteins based at least in part on the molecular descriptor matrix, wherein the machine learning model is trained by: accessing a training molecular descriptor matrix representing a training set of amino acid sequences corresponding to one or more empirically-evaluated proteins; and iteratively executing a process to refine a set of hyper-parameters associated with the machine learning model until a desired precision is reached, the process comprising: reducing the training molecular descriptor matrix by selecting one representative feature vector for each of a plurality of feature vector clusters, wherein each feature vector cluster includes similar feature vectors; determining one or more most-predictive feature vectors of the selected representative feature vectors for each feature vector cluster based on a correlation between the selected representative feature vectors and predetermined batch binding data associated with the one or more empirically-evaluated proteins; and calculating one or more cross-validation losses based at least in part on the one or more most-predictive feature vectors and the predetermined batch binding data; and updating the set of hyper-parameters based on the one or more cross-validation losses.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a diagram illustrating an experimental example for performing one or more protein purification processes as compared to a computational model-based example for performing one or more protein purification processes, in accordance with various embodiments.

FIG. 2 illustrates a high-level workflow diagram for performing feature generation, feature dimensionality reduction, regression model optimization, and model output-based feature selection, in accordance with various embodiments.

FIG. 3A illustrates a workflow diagram for optimizing hyper-parameters and learnable parameters of a machine learning model for performing one or more computational model-based protein purification processes, in accordance with various embodiments.

FIG. 3B illustrates workflow diagram for optimizing the machine learning model for performing one or more computational model-based protein purification processes, in accordance with various embodiments.

FIG. 4 illustrates a flow diagram of a method for generating a prediction of a molecular binding property of one or more target proteins as part of a streamlined process of protein purification for identifying target proteins, in accordance with various embodiments.

FIG. 5 illustrates an example computing system, in accordance with various embodiments.

FIG. 6 illustrates a diagram of an example artificial intelligence (AI) architecture included as part of the example computing system of FIG. 5, in accordance with various embodiments.

FIG. 7 illustrates another high-level workflow diagram for performing feature generation, feature dimensionality reduction, regression model optimization, and model output-based feature selection, in accordance with various embodiments.

FIG. 8 illustrates another workflow diagram for optimizing hyper-parameters and learnable parameters of a machine learning model for performing one or more computational model-based protein purification processes, in accordance with various embodiments.

FIG. 9 illustrates a process for training a machine learning model to predict a molecular binding property, in accordance with various embodiments.

FIGS. 10A-10D illustrate example plots illustrating how a principal component analysis can be used to predict a molecular binding property, in accordance with various embodiments.

FIGS. 11A-11F illustrate example heat maps illustrating a relationship between experimental conditions and experimental Kₚ values, and experimental conditions and modeled Kₚ values, respectively, in accordance with various embodiments.

FIG. 12 illustrates a flow diagram of a method for generating a prediction of a molecular binding property of one or more target proteins as part of another streamlined process of protein purification for identifying target proteins, in accordance with various embodiments.

### DESCRIPTION OF EXAMPLE EMBODIMENTS

Embodiments of the present embodiments are directed toward one or more computing devices, methods, and non-transitory computer-readable media that may utilize a machine learning model iteratively trained to generate a prediction of a molecular binding property of one or more target proteins as part of a streamlined process of protein purification for identifying target proteins (e.g., antibodies) and accelerating the selection process of therapeutic antibody candidates or other immunotherapy candidates by way of early identification of the most promising therapeutic antibody candidates. This streamlined process of identifying target proteins (e.g., antibodies) in-silico, for example, may facilitate and accelerate the downstream development and manufacturing of one or more therapeutic monoclonal antibodies (mAbs), bispecific antibodies (bsAbs), trispecific antibodies (tsAbs), or other similar immunotherapies that may be utilized to treat various diseases.

For example, once trained, the machine learning model (e.g., ensemble-learning model or a “boosting” ensemble-learning model) may be utilized to generate a prediction of a molecular binding property (e.g., a prediction of a percent protein bound at one or more specific pH values and specific salt concentrations and/or specific salt species and chromatographic resin) of one or more proteins by utilizing optimized hyper-parameters (e.g., general parameters, booster parameters, learning-task parameters) and learnable parameters (e.g., regression model weights, decision variables) learned during the training of the machine learning model and a selected k-best matrix of feature vectors of a molecular descriptor matrix representing a set of amino acid sequences corresponding to one or more proteins of interest.

Specifically, in accordance with the disclosed embodiments, once trained, the machine learning model may utilize the optimized hyper-parameters (e.g., general parameters, booster parameters, learning-task parameters) and learnable parameters (e.g., regression model weights, decision variables) learned during training to predict (i) a percent protein bound (e.g., a percentage of a set of proteins predicted to bind to a ligand within a solution) for a given pH value and salt concentration or a plurality of different combinations of pH values and salt concentrations, (ii) predict a percent protein bound (e.g., a percentage of a set of proteins predicted to bind to a ligand within a solution) for a set of pH values and salt concentrations, and/or (iii) predict a principal component (PC) representing a set of pH values and salt concentrations, for one or more target proteins based only on, as input, the selected k-best matrix of feature vectors of a molecular descriptor matrix representing a set of amino acid sequences corresponding to one or more proteins of interest and one or more sets of pH values and salt concentrations associated with the binding properties of the one or more proteins of interest.

In this way, by providing a computational-model-based prediction of the percent protein bound and/or the PC value for one or more proteins of interest, the molecular binding property and elution property of the one or more proteins of interest may be determined without considerable upstream experimentation. That is, desirable proteins of the one or more proteins of interest may be identified and distinguished from undesirable proteins of the one or more proteins of interest in-silico, and those desirable proteins identified in-silico may be further utilized to expedite and facilitate the downstream development of one or more therapeutic mAbs, bsAbs, tsAbs, or other similar immunotherapies that may be utilized to treat various diseases (e.g., by reducing upstream experimental duration and experimentation inefficiency and providing in-silico feedback on which candidate proteins may be difficult to purify, and, by extension, ultimately difficult to manufacture).

For example, during training of the machine learning model (e.g., “boosting” ensemble-learning model), the hyper-parameters (e.g., general parameters, booster parameters, learning-task parameters) and learnable parameters (e.g., regression model weights, decision variables) may be refined and optimized iteratively by 1) reducing a molecular descriptor matrix representing the set of amino acid sequences by clustering similar feature vectors of the molecular descriptor matrix based on a correlation distance metric, which may be calculated based on a Pearson's correlation, and discarding feature vectors other than the one closest to the cluster centroid; 2) determining the k-best most-predictive feature vectors of the reduced molecular descriptor matrix based on a k-best process and a correlation coefficient (e.g., maximum information coefficient (MIC)) for determining a correlation between the feature vectors of the reduced molecular descriptor matrix and an experimentally-determined percent protein bound for one or more specific pH values and salt concentrations; 3) calculating an n-number of cross-validation losses based on the k-best most-predictive feature vectors and the experimentally-determined percent protein bound; and 4) updating the hyper-parameters (e.g., general parameters, booster parameters, learning-task parameters) based on the n-number of cross-validation losses. In one or more examples, clustering of the one or more sets of descriptors may be based on a correlation distance between descriptors calculated from the Pearson's correlation (e.g., 1-abs (Pearson's correlation)).

In certain embodiments, reducing the molecular descriptor matrix, which may include a large set of amino acid sequence-based descriptors, by way of the foregoing feature dimensionality reduction and feature selection techniques may ensure that the regression model successfully converges to an accurately trained regression model as opposed to suffering overfitting due to superfluous or noisy descriptors. Further, it should be appreciated that, in some embodiments, alternative to utilizing the MIC as part of the determination of the correlation between the feature vectors of the reduced molecular descriptor matrix and the experimentally-determined percent protein bound, a distance correlation, mutual information, or other similar nonlinear correlation metric and/or a linear correlation metric (e.g., Pearson's correlation, f-statistic based metrics) may be utilized.

As used herein, terms “polypeptide” and “protein” may interchangeably refer to a polymer of amino acid residues, and are not limited to a minimum length. For example, such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition, for example. The terms “polypeptide” and “protein” may also include post-translational modifications of the polypeptide, for example, glycosylation, sialylation, acetylation, phosphorylation, and the like.

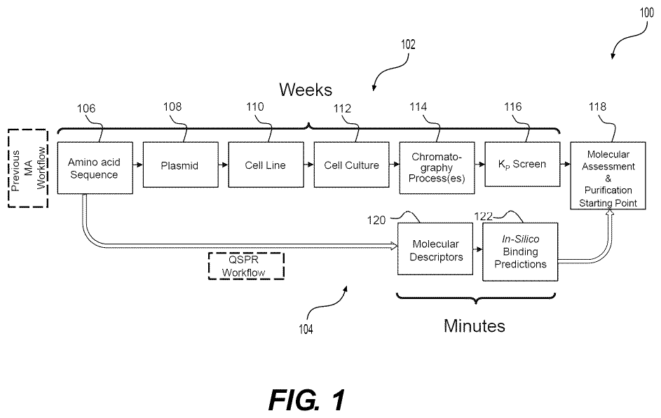

FIG. 1 illustrates a diagram 100 illustrating an experimental example 102 for performing one or more protein purification processes as compared to a computational model-based example 104 for performing one or more protein purification processes, in accordance with the disclosed embodiments. As illustrated, on the one hand, the experimental duration for the experimental example 102 for performing one or more protein purification processes may span a number of weeks. On the other hand, the execution time for the computational model-based example 104 for performing one or more protein purification processes may be only minutes.

For example, the experimental example 102 for performing one or more protein purification processes may include receiving amino acid sequences at block 106, selecting plasmids at block 108, engineering proteins by way of cell lines and cell cultures at blocks 110 and 112, respectively, performing one or more chromatography processes (e.g., an affinity chromatography process, ion exchange chromatography (IEX) process, a hydrophobic interaction chromatography (HIC) process, or a mixed-mode chromatography (MMC) process) at block 114, and performing a high throughput screening (HTS) and computing a partition coefficient (Kₚ) to quantify protein binding at block 116, all as part of a cumbersome and time-consuming protein purification process. A molecular assessment of one or more target proteins may be then performed at block 118.

In certain embodiments, in accordance with the presently-disclosed techniques, the computational model-based example 104 for performing one or more protein purification processes may include accessing amino acid sequences corresponding to one or more proteins of interest at block 106, generating a molecular descriptor matrix based on the amino acid sequences and reducing the molecular descriptor matrix at block 120, and utilizing a machine learning model (e.g., an ensemble-learning model) to generate a prediction of a molecular binding property of one or more target proteins at block 122, as part of an optimized and streamlined process of protein purification for identifying target proteins (e.g., antibodies) and accelerating the selection process of therapeutic antibody candidates or other immunotherapy candidates, in accordance with the presently disclosed embodiments.

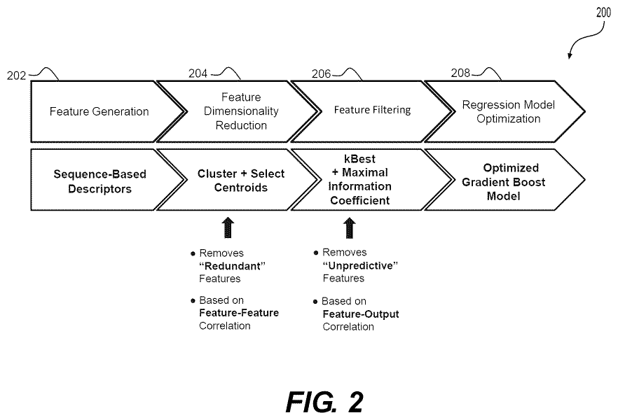

Indeed, as will be discussed in further detail below with respect to FIGS. 2-4, the machine learning model may utilize optimized hyper-parameters (e.g., general parameters, booster parameters, learning-task parameters) and learnable parameters (e.g., regression model weights, decision variables) learned during training to predict a percent protein bound (e.g., a percentage of a set of proteins predicted to bind to a ligand within a solution for a given pH value and salt concentration) for one or more target proteins based only on, as input, a selected k-best matrix of feature vectors of the molecular descriptor matrix generated at block 120 and one or more sets of pH values and salt concentrations associated with the binding properties of the one or more proteins of interest. The molecular assessment of the one or more target proteins may be then performed at block 118 without considerable upstream experimentation (e.g., as compared to the experimental example 102 for performing one or more protein purification processes). That is, desirable proteins of the one or more proteins of interest may be identified and distinguished from undesirable proteins of the one or more proteins of interest in-silico, and those desirable proteins identified in-silico may be further utilized to expedite and facilitate the downstream development of one or more therapeutic mAbs, bsAbs, tsAbs, or other similar immunotherapies that may be utilized to treat various diseases (e.g., by reducing upstream experimental duration and experimentation inefficiency and providing in-silico feedback on which candidate proteins may be difficult to purify, and, by extension, ultimately difficult to manufacture). As an example, based at least in part on the molecular descriptor matrix, the machine learning model may be configured to obtain a prediction of a molecular binding property of the one or more proteins. From the molecular binding property, desirable proteins may be identified.

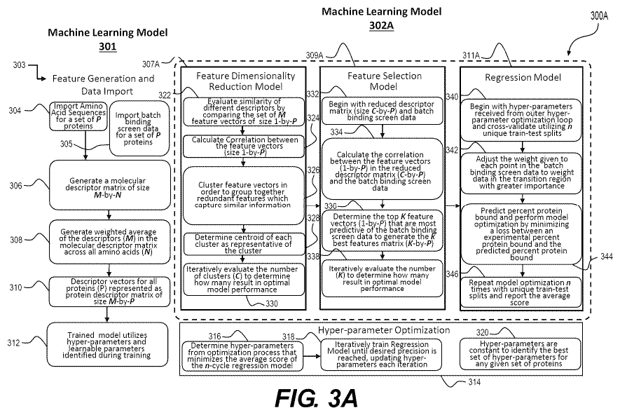

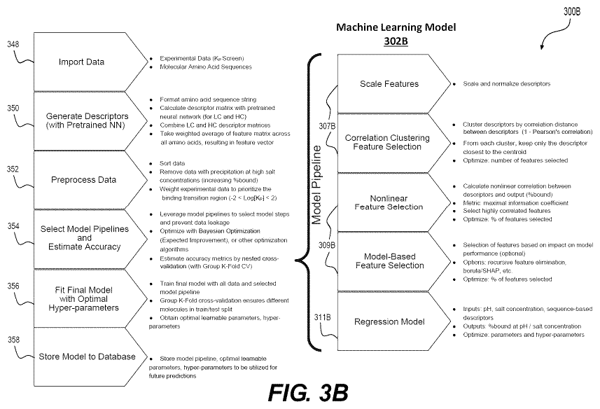

FIG. 2 illustrates a high-level workflow diagram 200 for performing feature generation 202, feature dimensionality reduction 204, model-output based feature selection 206, and regression model optimization 208, in accordance with the disclosed embodiments. Specifically, it should be appreciated that the high-level examples for performing feature generation 202, feature dimensionality reduction 204, model-output based feature selection 206, and regression model optimization 208, as discussed with respect to FIG. 2, may be discussed in greater detail below with respect to FIGS. 3A and 3B, and may be performed by a machine learning (e.g., a matrix generation machine learning model) in conjunction with another machine learning model (e.g., an ensemble-learning model) in accordance with the presently-disclosed embodiments.

That is, as discussed below with respect to FIGS. 3A and 3B, feature generation 202 may be performed by a machine learning model 301, feature dimensionality reduction 204 may be performed by a feature dimensionality reduction model 307A, 307B of a machine learning models 302A, 302B, model-output-based feature selection 206 may be performed by a feature selection model 309A, 309B of the machine learning models 302A, 302B, and regression model optimization 208 may be performed by a regression model 311A, 311B of the machine learning models 302A, 302B.

For example, as will be described in greater detail below, performing feature generation 202 may include generating, for example, 1024 molecular descriptors (e.g., amino acid sequence-based descriptors). In certain embodiments, performing feature dimensionality reduction 204 may include, for example, clustering and reducing the 1024 molecular descriptors (e.g., amino acid sequence-based descriptors) to remove redundant features or other features determined to be exceedingly similar. In certain embodiments, performing model-output-based feature selection 206 may include generating a k-best feature matrix to reduce the molecular descriptors to only the k-best most-predictive features of those molecular descriptors. As an example, the number of molecular descriptors may be 1024 based on the particular model used to generate the descriptors. As another example, the number of molecular descriptors may be greater or smaller, for instance, 2048 descriptors, 320 descriptors, etc. In certain embodiments, performing regression model optimization 208 may include, for example, optimizing hyper-parameters and learnable parameters associated with the regression model 311A, 311B of the machine learning models 302A, 302B. In certain embodiments, as will be discussed in greater detail below, the feature dimensionality reduction 204 and model-output-based feature selection 206 may, in some embodiments, be provided to filter the large set of amino acid sequence-based descriptors that may be generated as part of the feature generation 202. In this way, reducing the large set of amino acid sequence-based descriptors by way of feature dimensionality reduction 204 and model-output-based feature selection 206 may ensure that the regression model successfully converges to an accurately trained regression model as opposed to suffering overfitting due to superfluous or noisy descriptors.

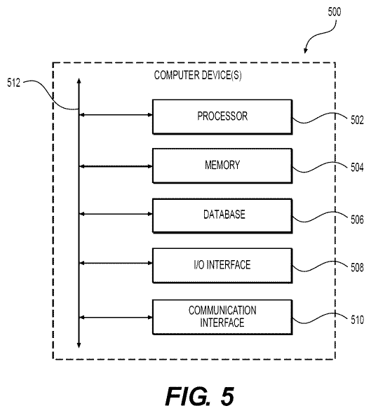

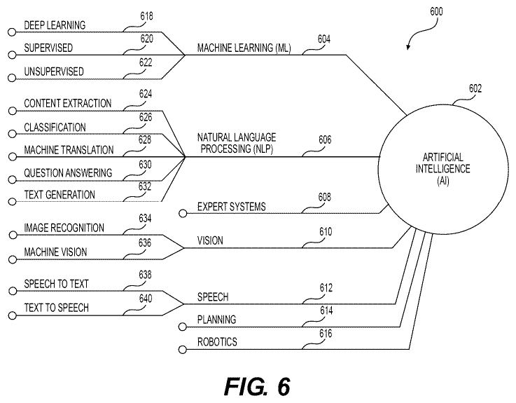

FIG. 3A illustrates a detailed workflow diagram 300A for optimizing hyper-parameters and learnable parameters of a machine learning model 302A (e.g., an ensemble-learning model) and utilizing the machine learning model 302A to generate a prediction of a molecular binding property of one or more target proteins as part of a streamlined process of protein purification for identifying target proteins (e.g., antibodies) and accelerating the selection process of therapeutic antibody or other immunotherapy candidates by way of early identification of the most promising therapeutic antibody candidates, in accordance with the disclosed embodiments. In certain embodiments, as depicted by FIG. 3A, the workflow diagram 300A may be performed in conjunction by a machine learning model 301 (e.g., a matrix generation machine learning model) and a machine learning model 302A (e.g., as illustrated by the dashed line) executed utilizing one or more processing devices (e.g., computing device(s) 500 and artificial intelligence architecture 600 to be discussed below with respect to FIGS. 5 and 6) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other processing device(s) that may be suitable for processing genomics data, proteomics data, metabolomics data, metagenomics data, transcriptomics data, or other omics data and making one or more decisions based thereon), software (e.g., instructions running/executing on one or more processors), firmware (e.g., microcode), or some combination thereof.

In certain embodiments, the machine learning model 302A may include, for example, any number of individual machine learning models or other predictive models (e.g., a feature dimensionality reduction model 307A, a feature selection model 309A, and a regression model 311A) that may be trained and executed in conjunction (e.g., trained and/or executed serially, in parallel, or end-to-end) to perform one or more predictions in sequence, such that the output of one or more initial models in the pipeline serves as the input to one or more succeeding models in the ensemble until a final overall prediction is outputted (e.g., “boosting”). For example, in some embodiments, the machine learning model 302A may include a gradient boosting model, an adaptive boosting (AdaBoost) model, an eXtreme gradient boosting (XGBoost) model, a light gradient boosted machine (LightGBM) model, or a categorical boosting (CatBoost) model.

In certain embodiments, as depicted by FIG. 3A and as will be discussed in greater detail below, the machine learning model 301 may perform one or more feature generation and data importing tasks 303, while the machine learning model 302A may include a feature dimensionality reduction model 307A, a feature selection model 309A, and a regression model 311A. One or more hyper-parameter optimization tasks 314 may further be performed to refine a set of hyper-parameters associated with the machine learning model 302A.

The workflow diagram 300A may begin at functional block 304 with the machine learning model 301 importing amino acid sequences for a set of one or more P proteins. For example, in certain embodiments, the machine learning model 301 may include one or more pre-trained artificial neural networks (ANNs), convolutional neural networks (CNNs), or other neural networks that may be suitable for generating a large set of amino acid sequence-based descriptors in, for example, a supervised, weakly-supervised, semi-supervised, or unsupervised manner. In accordance with the presently disclosed embodiments, the amino acid sequence-based descriptors may be utilized (e.g., as opposed to structure-based descriptors), as the amino acid sequence-based descriptors may be more effective for training the machine learning model 302A to generate predictions of the molecular binding property of one or more target proteins (e.g., as compared to utilizing structure-based descriptors). Indeed, as will be discussed in greater detail below, the feature dimensionality reduction model 307A, 307B and the feature selection model 309A, 309B may, in some embodiments, be provided to filter the large set of amino acid sequence-based descriptors that may be outputted by the machine learning model 301. In this way, reducing the large set of amino acid sequence-based descriptors by way of the feature dimensionality reduction model 307A, 307B and the feature selection model 309A, 309B may ensure that the regression model 311A, 311B successfully converges to an accurately trained regression model as opposed to suffering overfitting due to superfluous or noisy descriptors.

At functional block 305, predetermined batch binding data for the set of one or more P proteins may also be imported for use by the machine learning model 302A. For example, in certain embodiments, the predetermined batch binding data may include an experimentally-determined percent protein bound for one or more specific pH values and salt concentrations (e.g., a sodium-chloride (NaCl) concentration, a phosphate (PO₄³⁻) concentration) and/or salt species (e.g., sodium acetate (CH3COONa) species, a sodium phosphate (Na3PO4) species) and chromatographic resin.

The workflow diagram 300A may then continue at functional block 306 with the machine learning model 301 generating a molecular descriptor matrix of size M-by-N. For example, in certain embodiments, from the amino acid sequence of each protein of the set of one or more P proteins, the machine learning model 301 (e.g., neural network, convolutional neural network (CNN), deep neural network (DNN)) may generate a molecular descriptor matrix of size M-by-N, where M is the number of descriptors (M=1024) and N is the number of amino acids in a given protein of the set of one or more P proteins.

In certain embodiments, the workflow diagram 300A may then continue at functional block 308 with generating a weighted average of the descriptors (M) in the molecular descriptor matrix across all amino acids (N). For example, in certain embodiments, a weighted average of the descriptors (M) in the molecular descriptor matrix across all amino acids (N) may be calculated, resulting in a descriptor vector of size M-by-1 for each protein of the set of one or more P proteins. For example, in some embodiments, the machine learning model 301 may generate one or more M-by-1 vectors of descriptors for each protein of the set of one or more P proteins. In certain embodiments, the workflow diagram 300A may then continue at functional block 310 with representing descriptor vectors for all proteins (P) as a protein descriptor matrix of size M-by-P.

In certain embodiments, functional block 312 of the workflow diagram 300A may illustrate an iteration of the machine learning model 302A having already been trained, in which a set of hyper-parameters (e.g., general parameters, booster parameters, learning-task parameters) and a set of learnable parameters (e.g., regression model weights, decision variables) were identified during the training of the machine learning model 302A. Specifically, as part of the one or more hyper-parameter optimization tasks 314 that are performed with respect to the machine learning model 302A, at functional block 316, hyper-parameters that minimize the average score of an n-cycle regression-based model of the machine learning model 302A are determined.

For example, in one embodiment, a baseline set of hyper-parameters (e.g., general parameters, booster parameters, learning-task parameters) may be selected and then updated iteratively so as to minimize the average score of the 10-cycle regression-based model of the machine learning model 302A. In certain embodiments, at functional block 318, the machine learning model 302A may be iteratively trained until a desired precision is reached, refining a set of hyper-parameters by updating the selected hyper-parameters (e.g., general parameters, booster parameters, learning-task parameters) with each successive iteration. The selected hyper-parameters may be updated based on one or more cross-validation losses. For example, in one embodiment, the desired precision is reached when a given set of hyper-parameters selected minimizes (e.g., reaches lowest possible value or error on a scale of 0.0 to 1.0) the one or more cross-validation losses. For example, as will be further described below, in some embodiments, minimizing the one or more cross-validation losses may include minimizing a loss between a predicted percent protein bound and an experimentally-determined percent protein bound. Thus, in some embodiments, the desired precision of the machine learning model 302A is reached when a given set of hyper-parameters selected minimizes the loss between the predicted percent protein bound and the experimentally-determined percent protein bound.

In accordance with the presently-disclosed techniques, in certain embodiments, at functional block 320, the hyper-parameters may be optimized by evaluating a cross-validation loss function based on the k-best feature vectors most-predictive of the predetermined batch binding data, the predetermined batch binding data (e.g., experimentally-determined percent protein bound for one or more specific pH values and salt concentrations and/or salt species and chromatographic resin), the baseline set of hyper-parameters (e.g., general parameters, booster parameters, learning-task parameters), and a set of learnable parameters (e.g., regression model weights, decision variables) associated with, and determined by, the machine learning model 302A. The machine learning model 302A may then minimize the cross-validation loss function by varying the set of learnable parameters while the k-best most-predictive feature vectors, the predetermined batch binding data, and the set of hyper-parameters remain constant.

In certain embodiments, as previously noted above, the machine learning model 302A may include a feature dimensionality reduction model 307A, a feature selection model 309A, and a regression model 311A. A feature dimensionality reduction task may reduce the molecular descriptor matrix by selecting one representative feature vector for each of a plurality of feature vector clusters. Beginning with the feature dimensionality reduction model 307A, in certain embodiments, the workflow diagram 300A may continue at functional block 322 with the machine learning model 302A evaluating a similarity of different descriptors by comparing the set of M feature vectors of size 1-by-P.

For example, in certain embodiments, for the protein descriptor matrix (M-by-P), the similarity of different descriptors may be evaluated by comparing the set of M feature vectors of size 1-by-P. In certain embodiments, the workflow diagram 300A may then continue at functional block 324 with the machine learning model 302A calculating a correlation between the feature vectors (size 1-by-P). For example, in certain embodiments, the machine learning model 302A may calculate a correlation distance metric, which may, for example, be calculated using a Pearson's correlation, between each of the feature vectors (size 1-by-P). In one or more examples, clustering of the descriptors may be based on the correlation distance between the descriptors calculated from the Pearson's correlation (e.g., 1-abs (Pearson's correlation)).

In certain embodiments, the workflow diagram 300A may then continue at functional block 326 with the machine learning model 302A clustering feature vectors in order to group together redundant features that capture similar information. For example, in certain embodiments, utilizing an agglomerative-clustering process and the calculated distance correlation metric, which may be calculated based on the Pearson's correlation, the machine learning model 302A may cluster feature vectors in order to group together any and all redundant features that include similar information (similar feature vectors). In certain embodiments, the workflow diagram 300A may then continue at functional block 328 with the machine learning model 302A determining a centroid of each cluster as representative of the cluster, which is valuable for feature selection. Additionally, the selection of the centroid of each cluster can enable a set of orthogonal features to be selected, which can reduce multicollinearity. In certain embodiments, the workflow diagram 300A may then continue at functional block 330 with the machine learning model 302A iteratively evaluating the number of clusters (C) to determine how many result in optimal performance of the machine learning model 302A.

In certain embodiments, the machine learning model 302A may also include the feature selection model 309A, which may determine one or more most-predictive feature vectors of the selected representative feature vectors for each feature vector cluster. The workflow diagram 300A may continue at functional block 332 with the machine learning model 302A, starting with the reduced descriptor matrix (size C-by-P), calculating a correlation between the feature vectors (1-by-P) in the reduced descriptor matrix (C-by-P) and the predetermined batch binding data at functional block 334. For example, in certain embodiments, utilizing a nonlinear correlation metric (e.g., maximal information coefficient (MIC), distance correlation, mutual information, or other similar nonlinear correlation metric), and/or utilizing a linear correlation metric (e.g., a Pearson's correlation), the machine learning model 302A may calculate the correlation between the selected representative feature vectors (1-by-P) in the reduced descriptor matrix (C-by-P) and the predetermined batch binding data (associated with the one or more proteins) in order to rank which features and/or descriptors capture information that is suitable for predicting the outputs.

In certain embodiments, the workflow diagram 300A may then continue at functional block 336 with the machine learning model 302A determining the top K feature vectors (1-by-P) that are most predictive of the predetermined batch binding data to generate the k-best features matrix (K-by-P). For example, in certain embodiments, utilizing a k-best process, the machine learning model 302A may select the top K feature vectors (1-by-P) that are most predictive of the predetermined batch binding data (e.g., as scored by the MIC, distance correlation, mutual information, or other similar nonlinear correlation metric) to generate a k-best features matrix (K-by-P). Specifically, in one embodiment, the k-best features matrix (K-by-P) may maintain the top K feature vectors (1-by-P), where K is an integer value indicating a number of the feature vectors that are maintained. In another embodiment, the k-best features matrix (K-by-P) may maintain the top K feature vectors, where K is a percentage value indicating a percentage of the feature vectors that are maintained. In certain embodiments, the workflow diagram 300A may then continue at functional block 338 with the machine learning model 302A iteratively evaluating the K feature vectors to determine how many result in optimal performance of the machine learning model 302A.

In certain embodiments, the machine learning model 302A may also include the regression model 311. For example, the workflow diagram 300A may continue at functional block 340 with the machine learning model 302A, starting with the baseline hyper-parameters selected and updated as part of the hyper-parameter optimization tasks 314, the machine learning model 302A may then perform cross-validation utilizing n unique train-test splits (e.g., Group K-Fold cross-validation, stratified K-Fold cross-validation). The cross-validation may include calculating one or more cross-validation losses based at least in part on the one or more most-predictive feature vectors and the predetermined batch binding data.

For example, starting with the baseline hyper-parameters selected and updated as part of the hyper-parameter optimization tasks 314, the k-best features matrix, and the predetermined batch binding data, the machine learning model 302A may perform cross-validation utilizing 10 unique train-test splits of the k-best features matrix and the predetermined batch binding data (e.g., training data set). In one embodiment, the machine learning model 302A may perform cross-validation utilizing 2 or more, 5 or more, 10 or more, or other quantities of, unique train-test splits of the k-best features matrix and the predetermined batch binding data (e.g., training data set) in order to, for example, reduce a possibility of overfitting or miscalculating the accuracy of the machine learning model 302A due to the train-test split. In other embodiments, the machine learning model 302A may perform cross-validation utilizing any n integer number of unique train-test splits, so long as the integer number n is less than or equal to a number of data points corresponding, for example, to the training dataset.

In certain embodiments, the workflow diagram 300A may then continue at functional block 342 with the machine learning model 302A adjusting the weight given to the data of the predetermined batch binding data (e.g., percent protein bounds at various pH values and salt concentrations and/or salt species and chromatographic resin) to the weight data in the transition region with greater importance. For example, the machine learning model 302A may adjust the weight given to each point in the predetermined batch binding data to weight data in the transition region (e.g., partially bound proteins) with more importance than those that are fully-bound proteins or fully-unbound. In certain embodiments, the workflow diagram 344 with the machine learning model 302A predicting a percent protein bound for the set of proteins P and optimizing the machine learning model 302A by minimizing a loss between the predicted percent protein bound and an experimentally-determined percent protein bound. In certain embodiments, the workflow diagram 300A may then continue at functional block 346 with the machine learning model 302 repeating model optimization n times with unique train-test splits and reporting the average score.

Specifically, the regression tasks of the machine learning model 302A may include receiving the predetermined batch binding data and the k-best features matrix and predicting (at functional block 346) a percent protein bound for the set of proteins P based on the predetermined batch binding data and the k-best features matrix. The machine learning model 302A may be then optimized by minimizing (at functional block 346) a loss (e.g., sum of squared error (SSE)) between the predicted percent protein bound and the experimentally-determined percent protein bound for one or more specific pH values and salt concentrations (e.g., a sodium-chloride (NaCl) concentration, a phosphate (PO₄³⁻) concentration) and/or salt species (e.g., a sodium acetate (CH3COONa) species, a sodium phosphate (Na3PO4) species) and chromatographic resin. In some embodiments, the pH value and salt concentration and/or salt species and chromatographic resin may be associated with the molecular binding property of the one or more proteins.

Accordingly, as set forth by the workflow diagram 300A of FIG. 3A, a machine learning model 302A may be iteratively trained to generate a prediction of a molecular binding property of one or more target proteins as part of a streamlined process of protein purification for identifying target proteins (e.g., antibodies) and accelerating the selection process of therapeutic antibody candidates or other immunotherapy candidates by way of early identification of the most promising therapeutic antibody candidates. The streamlined process of identifying target proteins (e.g., antibodies) in-silico, for example, may facilitate and accelerate the downstream development and manufacturing of one or more therapeutic mAbs, bsAbs, tsAbs, 2+1 Abs, or other similar immunotherapies that may be utilized to treat various diseases.

For example, once trained, the machine learning model 302A (e.g., “boosting” machine learning model) may be utilized to generate a prediction of a molecular binding property (e.g., a prediction of a percent protein bound at one or more specific pH values and specific salt concentrations and/or specific salt species and chromatographic resin) of one or more proteins by utilizing optimized hyper-parameters (e.g., general parameters, booster parameters, learning-task parameters) and learnable parameters (e.g., regression model weights, decision variables) learned during the training of the machine learning model 302A and a selected k-best matrix of feature vectors of a molecular descriptor matrix representing a set of amino acid sequences corresponding to one or more proteins of interest.

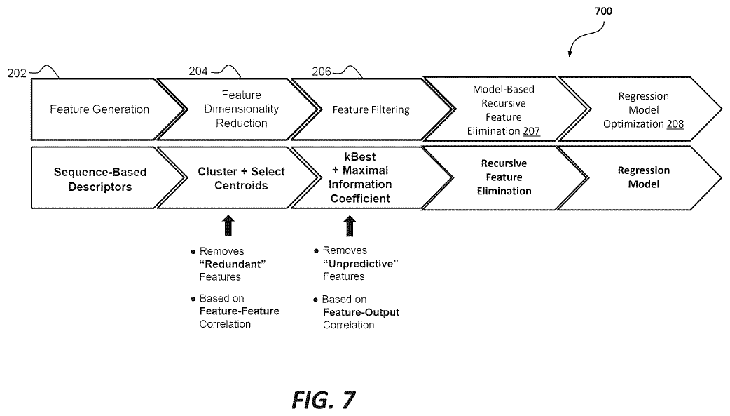

Specifically, in accordance with the disclosed embodiments, once trained, the machine learning model 302A may utilize the optimized hyper-parameters (e.g., general parameters, booster parameters, learning-task parameters) and learnable parameters (e.g., regression model weights, decision variables) learned during training to predict a percent protein bound (e.g., a percentage of a set of proteins predicted to bind to a ligand within a solution for a given pH value and salt concentration) and/or a first principal component (PC1) of the Log (Kₚ) values (logit transform of percent bound) for one or more target proteins based only on, as input, the selected k-best matrix of feature vectors of a molecular descriptor matrix representing a set of amino acid sequences corresponding to one or more proteins of interest and one or more sets of pH values and salt concentrations and/or salt species and chromatographic resin associated with the binding properties of the one or more proteins of interest. In some embodiments, as described below with reference to FIGS. 7-13, instead of predicting the percent bound for a given pH and salt concentration, a first principal component (PC1) of the Log (Kₚ) values (logit transform of percent bound) may be predicted from data across the design space (some set of datapoint covering a range of pH/salt concentrations) for a given resin.

In this way, by providing a computational-model-based prediction of the percent protein bound for one or more proteins of interest, the molecular binding property and elution property of the one or more proteins of interest may be determined without considerable upstream experimentation. That is, desirable proteins of the one or more proteins of interest may be identified and distinguished from undesirable proteins of the one or more proteins of interest in-silico, and those desirable proteins identified in-silico may be further utilized to expedite and facilitate the downstream development of one or more therapeutic mAbs, bsAbs, tsAbs, or other similar immunotherapies that may be utilized to treat various diseases (e.g., by reducing upstream experimental duration and experimentation inefficiency and providing in-silico feedback on which candidate proteins may be difficult to purify, and, by extension, ultimately difficult to manufacture). As an example, based at least in part on the molecular descriptor matrix, the machine learning model may be configured to obtain a prediction of a molecular binding property of the one or more proteins. From the molecular binding property, desirable proteins may be identified. While the present embodiments are discussed herein primarily with respect to the machine learning model 302A generating a prediction of a molecular binding property of one or more target proteins, it should be appreciated that the machine learning model 302A as trained may also generate a prediction of an elution property of the one or more proteins or generate a prediction of a flow-through property of the one or more proteins, in accordance with the presently disclosed embodiments.

FIG. 3B illustrates a detailed workflow diagram 300B for optimizing the machine learning model 302A as discussed above with respect to FIG. 3A and utilizing the optimized machine learning model 302B to generate a prediction of a molecular binding property of one or more target proteins as part of a streamlined process of protein purification for identifying target proteins (e.g., antibodies) and accelerating the selection process of therapeutic antibody candidates or other immunotherapy candidates by way of early identification of the most promising therapeutic antibody candidates, in accordance with the disclosed embodiments. Specifically, as will be further appreciated below, the workflow diagram 300B may represent an improvement over the workflow diagram 300A as discussed above with respect to FIG. 3A. For example, as discussed below, the workflow diagram 300B may include performing one or more Bayesian optimization processes (e.g., sequential model-based optimization (SMBO), expected improvement (EI)) to iteratively optimize and evaluate the machine learning model 302B by, for example, selectively determining which of the functional blocks of feature dimensionality reduction model 307B, feature selection model 309B, and regression model 311B to execute, as well as the order in which the determined functional blocks of feature dimensionality reduction model 307B, feature selection model 309B, and regression model 311B are to be executed.

In certain embodiments, as depicted by FIG. 3B, the workflow diagram 300B may be performed utilizing one or more processing devices (e.g., computing device(s) 500 and artificial intelligence architecture 600 to be discussed below with respect to FIGS. 5 and 6) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other processing device(s) that may be suitable for processing genomics data, proteomics data, metabolomics data, metagenomics data, transcriptomics data, or other omics data and making one or more decisions based thereon), software (e.g., instructions running/executing on one or more processors), firmware (e.g., microcode), or some combination thereof.

In certain embodiments, the workflow diagram 300B may begin at functional block 348 with importing amino acid sequences for a set of one or more P proteins. For example, in some embodiments, at functional block 348, one or more partition coefficient (Kₚ) screens of experimental amino acid sequences for a set of one or more P proteins and/or molecular amino acid sequences for a set of one or more P proteins may be imported. The workflow diagram 300B may then continue at functional block 350 with formatting the amino acid sequences for the set of one or more P proteins and generating a molecular descriptor matrix of size M-by-N.

In certain embodiments, M may be the number of descriptors (M=1024) and N may be the number of amino acids in a given protein of the set of one or more P proteins for both light-chain (LC) and heavy-chain (HC) amino acid sequences. At functional block 350, the workflow diagram 300B may also include generating a weighted average of the descriptors (M) in the molecular descriptor matrix across all amino acids (N). For example, in certain embodiments, a weighted average of the descriptors (M) in the molecular descriptor matrix across all amino acids (N) may be calculated, resulting in a descriptor vector of size M-by-1 for each protein of the set of one or more P proteins. For example, in some embodiments, the machine learning model 301 (as described above with respect to FIG. 3A) may generate one or more M-by-1 vectors of descriptors for each protein of the set of one or more P proteins.

In certain embodiments, the workflow diagram 300B may then continue at functional block 352 with preprocessing the descriptor vector by removing amino acid sequence data with precipitation at high salt concentrations and weighting experimental data to prioritize the binding transition region (e.g., −2<Log [Kₚ]<+2, or −0.5<Log [Kₚ]<+2). In certain embodiments, as previously discussed, the workflow diagram 300B may be provided for optimizing the machine learning model 302A as discussed above with respect to FIG. 3A, and then the optimized machine learning model 302B may be utilized to generate a prediction of a molecular binding property of one or more target proteins in accordance with the presently-disclosed embodiments. For example, the workflow diagram 300B may continue at functional block 354 selectively determining which of the functional blocks of feature dimensionality reduction model 307B, feature selection model 309B, and regression model 311B to execute, as well as the order in which the functional blocks of feature dimensionality reduction model 307B, feature selection model 309B, and regression model 311B are to be executed.

For example, in certain embodiments, as part of a process for optimizing the machine learning model 302B (e.g., an ensemble-learning model), the workflow diagram 300B at functional block 354 may perform one or more Bayesian optimization processes (e.g., sequential model-based optimization (SMBO), expected improvement (EI)) to optimize and evaluate the machine learning model 302B. For example, in one embodiment, the Bayesian optimization processes (e.g., SMBO, EI) may include, for example, one or more probability-based objective functions that may be constructed and utilized to select the most predictive or the most promising of the functional blocks of feature dimensionality reduction model 307B, feature selection model 309B, and regression model 311B to execute and/or the order in which to execute these functional blocks. These functional blocks of the feature dimensionality reduction model 307B, the feature selection model 309B, and the regression model 311B are discussed below.

In certain embodiments, based on the functional blocks of feature dimensionality reduction model 307B, feature selection model 309B, and regression model 311B selected for execution, the workflow diagram 300B at functional block 354 may further proceed in estimating the accuracy of the machine learning model 302B utilizing, for example, nested cross-validation with Group K-Fold cross-validation. In this way, the workflow diagram 300B may optimize the machine learning model 302B to more efficiently (e.g., decreasing the execution time of the machine learning model 302B and database capacity suitable for storing the machine learning model 302B) generate a prediction of a molecular binding property of one or more target proteins as compared to, for example, the machine learning model 302A as discussed above with respect to FIG. 3A.

In certain embodiments, the workflow diagram 300B may then continue at functional block 356 with training and evaluating the optimized machine learning model 302B. For example, in some embodiments, the optimized machine learning model 302B (e.g., as optimized at functional block 354) may be trained and evaluated based on the descriptor vector representing the amino acid sequences for the set of one or more P proteins (e.g., as computed at functional block 352) and the functional blocks of feature dimensionality reduction model 307B, feature selection model 309B, and regression model 311B selected for execution.

In certain embodiments, the workflow diagram 300B at functional block 356 may further include applying the optimized set of hyper-parameters (e.g., general parameters, booster parameters, learning-task parameters) and optimized set of learnable parameters (e.g., regression model weights, decision variables) (e.g., as iteratively optimized and discussed above with respect to the workflow diagram 300A of FIG. 3A) to the optimized machine learning model 302B and utilizing the optimized machine learning model 302B to generate a prediction of a molecular binding property of one or more target proteins in accordance with the presently-disclosed embodiments. In certain embodiments, the workflow diagram 300B may then conclude at functional block 358 with storing the optimized machine learning model 302B, the optimized set of hyper-parameters (e.g., general parameters, booster parameters, learning-task parameters), and the optimized set of learnable parameters (e.g., regression model weights, decision variables) to be utilized for subsequent predictions of the molecular binding property of one or more target proteins.

In certain embodiments, for example, during the inference phase (e.g., after the optimized machine learning model 302B is trained and stored along with the optimized set of hyper-parameters and learnable as described above), as further illustrated by FIG. 3B, the feature dimensionality reduction model 307B of the machine learning model 302B may receive or import a molecular descriptor matrix and scale and normalize one or more sets of the descriptors of the descriptor matrix. For example, the molecular descriptor matrix may represent a set of amino acid sequences corresponding to a set of P proteins. In certain embodiments, the feature dimensionality reduction model 307B may then perform a clustering of the one or more sets of descriptors by determining a correlation distance between descriptors (e.g., 1-abs (Pearson's correlation)), and then only the descriptors closest to the centroid may be stored. For example, in some embodiments, utilizing the calculated correlation distance metric, which may be calculated based on the Pearson's correlation, the feature dimensionality reduction model 307B may cluster feature vectors in order to group together any and all redundant features that include similar information (similar feature vectors) and determine a centroid of each cluster as representative of the cluster. In certain embodiments, the feature dimensionality reduction model 307B may then optimize the number of descriptors selected.

In certain embodiments, utilizing a MIC metric, a distance correlation, mutual information, or other similar nonlinear correlation metric, and/or other linear correlation metrics (e.g., Pearson's correlation), the feature selection model 309B may then calculate a nonlinear correlation between descriptors and output a percent protein bound. In one or more other embodiments, the feature selection model 309B may calculate a nonlinear correlation between descriptors and output a percent protein bound utilizing distance correlation, mutual information, or other similar nonlinear correlation metric. For example, the feature selection model 309B may determine the k-best most-predictive feature vectors of the reduced molecular descriptor matrix based on a k-best process and the MIC for determining a correlation between the feature vectors of the reduced molecular descriptor matrix and an experimentally-determined percent protein bound for one or more specific pH values and salt concentrations and/or salt species and chromatographic resin. In other embodiments, alternative to utilizing the MIC as part of the determination of the correlation, a distance correlation, mutual information, or other similar nonlinear correlation metric may be utilized. The feature selection model 309B may then select the highly correlated descriptors and optimize the selected descriptors. In certain embodiments, the feature selection model 309B may then select a set of descriptors based on impact to the overall performance (e.g., processing speed, storage capacity) of the machine learning model 302B. For example, in some embodiments, the feature selection model 309B may iteratively evaluating the K descriptors to determine how many result in optimal performance of the machine learning model 302B. In some embodiments, the feature selection model 309B may perform the selection of the set of descriptors based on impact to the overall performance entirely selectively.

For example, in other embodiments, the feature selection model 309B may perform, for example, one or more Boruta feature selection algorithms, one or more SHapley Additive explanations (SHAP) feature selection algorithms, or other similar recursive feature elimination algorithm to select the K descriptors and to optimize the percentage of the number of selected K descriptors. In certain embodiments, the regression model 311B of the machine learning model 302B may then receive as inputs a pH value, a salt concentration, and the descriptors sequence-based descriptors, and may then output a prediction of a percent protein bound for the set of proteins P and optimizing the machine learning model 302B by minimizing a loss between the predicted percent protein bound and an experimentally-determined percent protein bound.

Accordingly, as further set forth by the workflow diagram 300B of FIG. 3B, a machine learning model 302B iteratively trained to generate a prediction of a molecular binding property of one or more target proteins as part of a streamlined process of protein purification for identifying target proteins and accelerating the selection process of therapeutic antibody candidates or other immunotherapy candidates by way of early identification of the most promising therapeutic antibody candidates. The streamlined process of identifying target proteins (e.g., antibodies) in-silico, for example, may facilitate and accelerate the downstream development and manufacturing of one or more therapeutic mAbs, bsAbs, tsAbs, or other similar immunotherapies that may be utilized to treat various patient diseases.

For example, once trained, the machine learning model 302B (e.g., “boosting” machine learning model) may be utilized to generate a prediction of a molecular binding property (e.g., a prediction of a percent protein bound at one or more specific pH values and specific salt concentrations and/or specific salt species and chromatographic resin) of one or more proteins by utilizing optimized hyper-parameters (e.g., general parameters, booster parameters, learning-task parameters) and learnable parameters (e.g., regression model weights, decision variables) learned during the training of the machine learning model 302B and a selected k-best matrix of feature vectors of a molecular descriptor matrix representing a set of amino acid sequences corresponding to one or more proteins of interest.

Specifically, in accordance with the disclosed embodiments, once trained, the machine learning model 302B may utilize the optimized hyper-parameters (e.g., general parameters, booster parameters, learning-task parameters) and learnable parameters (e.g., regression model weights, decision variables) learned during training to predict a percent protein bound (e.g., a percentage of a set of proteins predicted to bind to a ligand within a solution for a given pH value and salt concentration) for one or more target proteins based only on, as input, the selected k-best matrix of feature vectors of a molecular descriptor matrix representing a set of amino acid sequences corresponding to one or more proteins of interest and one or more sets of pH values and salt concentrations and/or salt species and chromatographic resin associated with the binding properties of the one or more proteins of interest. Additionally, in accordance with the disclosed embodiments, the ensemble-learning 302B may be further optimized utilizing one or more Bayesian optimization processes to more efficiently to generate the prediction of the molecular binding property (e.g., a prediction of a percent protein bound at one or more specific pH values and specific salt concentrations and/or specific salt species and chromatographic resin).

In this way, by providing a computational-model-based and optimized prediction of the percent protein bound for one or more proteins of interest, the molecular binding property and elution property of the one or more proteins of interest may be determined without considerable upstream experimentation. That is, desirable proteins of the one or more proteins of interest may be identified and distinguished from undesirable proteins of the one or more proteins of interest in-silico, and those desirable proteins identified in-silico may be further utilized to expedite and facilitate the downstream development of one or more therapeutic mAbs, bsAbs, tsAbs, or other similar immunotherapies that may be utilized to treat various diseases (e.g., by reducing upstream experimental duration and experimentation inefficiency and providing in-silico feedback on which candidate proteins may be difficult to purify, and, by extension, ultimately difficult to manufacture). As an example, based at least in part on the molecular descriptor matrix, the machine learning model may be configured to obtain a prediction of a molecular binding property of the one or more proteins. From the molecular binding property, desirable proteins may be identified. While the present embodiments are discussed herein primarily with respect to the machine learning model 302B generating a prediction of a molecular binding property of one or more target proteins, it should be appreciated that the machine learning model 302B as trained may also generate a prediction of an elution property of the one or more proteins or generate a prediction of a flow-through property of the one or more proteins, in accordance with the presently disclosed embodiments.

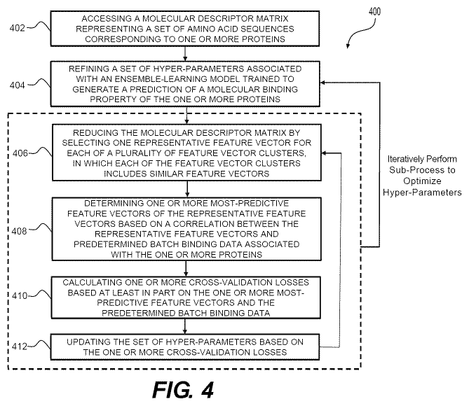

FIG. 4 illustrates a flow diagram of a method 400 for generating a prediction of a molecular binding property of one or more target proteins as part of a streamlined process of protein purification for identifying target proteins (e.g., antibodies) and accelerating the selection process of therapeutic antibody candidates or other immunotherapy candidates by way of early identification of the most promising therapeutic antibody candidates, in accordance with the disclosed embodiments. The method 400 may be performed utilizing one or more processing devices (e.g., computing device(s) and artificial intelligence architecture to be discussed below with respect to FIGS. 5 and 6) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other processing device(s) that may be suitable for processing genomics data, proteomics data, metabolomics data, metagenomics data, transcriptomics data, or other omics data and making one or more processors), firmware (e.g., microcode), or some combination thereof.

The method 400 may begin at block 402 with one or more processing devices accessing a molecular descriptor matrix representing a set of amino acid sequences corresponding to one or more proteins. The method 400 may then continue at block 404 with one or more processing devices refining a set of hyper-parameters associated with a machine learning model trained to generate a prediction of a molecular binding property of the one or more proteins. As illustrated, the method 400 may then proceed with an iterative sub-process of optimizing the set of hyper-parameters by iteratively executing the sub-process (e.g., illustrated by the dashed lines around a portion of the method 400 of FIG. 4) until a desired precision is reached for the machine learning model.

For example, the method 400 may continue at block 406 with one or more processing devices reducing the molecular descriptor matrix by selecting one representative feature vector for each of a plurality of feature vector clusters, wherein each of the feature vector clusters includes similar feature vectors. The method 400 may then continue at block 408 with one or more processing devices determining one or more most-predictive feature vectors of the selected representative feature vectors for each of the plurality of feature vector clusters based on a correlation between the selected representative feature vectors and predetermined batch binding data associated with the one or more proteins. The method 400 may then continue at block 410 with one or more processing devices calculating one or more cross-validation losses based at least in part on the one or more most-predictive feature vectors and the predetermined batch binding data. The method 400 may then conclude at block 412 with one or more processing devices updating the set of hyper-parameters based on the one or more cross-validation losses.

FIG. 5 illustrates an example of one or more computing device(s) 500 that may be utilized to generate a prediction of a molecular binding property of one or more target proteins as part of a streamlined process of protein purification for identifying target proteins (e.g., antibodies) and accelerating the selection process of therapeutic antibody candidates or other immunotherapy candidates by way of early identification of promising therapeutic antibody candidates, in accordance with the disclosed embodiments. In certain embodiments, the one or more computing device(s) 500 may perform one or more steps of one or more methods described or illustrated herein. In certain embodiments, the one or more computing device(s) 500 provide functionality described or illustrated herein. In certain embodiments, software running on the one or more computing device(s) 500 performs one or more steps of one or more methods described or illustrated herein, or provides functionality described or illustrated herein. Certain embodiments include one or more portions of the one or more computing device(s) 500.

This disclosure contemplates any suitable number of computing systems 500. This disclosure contemplates one or more computing device(s) 500 taking any suitable physical form. As example and not by way of limitation, one or more computing device(s) 500 may be an embedded computer system, a system-on-chip (SOC), a single-board computer system (SBC) (e.g., a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, the one or more computing device(s) 500 may be unitary or distributed; span multiple locations; span multiple machines; span multiple data centers; or reside in a cloud, which may include one or more cloud components in one or more networks.

Where appropriate, the one or more computing device(s) 500 may perform without substantial spatial or temporal limitation one or more steps of one or more methods described or illustrated herein. As an example, and not by way of limitation, the one or more computing device(s) 500 may perform, in real-time or in batch mode, one or more steps of one or more methods described or illustrated herein. The one or more computing device(s) 500 may perform, at different times or at different locations, one or more steps of one or more methods described or illustrated herein, where appropriate.

In certain embodiments, the one or more computing device(s) 500 includes a processor 502, memory 504, database 506, an input/output (I/O) interface 508, a communication interface 510, and a bus 512. Although this disclosure describes and illustrates a particular computer system having a particular number of particular components in a particular arrangement, this disclosure contemplates any suitable computer system having any suitable number of any suitable components in any suitable arrangement. In certain embodiments, processor 502 includes hardware for executing instructions, such as those making up a computer program. As an example, and not by way of limitation, to execute instructions, processor 502 may retrieve (or fetch) the instructions from an internal register, an internal cache, memory 504, or database 506; decode and execute them; and then write one or more results to an internal register, an internal cache, memory 504, or database 506. In certain embodiments, processor 502 may include one or more internal caches for data, instructions, or addresses. This disclosure contemplates processor 502 including any suitable number of any suitable internal caches, where appropriate. As an example, and not by way of limitation, processor 502 may include one or more instruction caches, one or more data caches, and one or more translation lookaside buffers (TLBs). Instructions in the instruction caches may be copies of instructions in memory 504 or database 506, and the instruction caches may speed up retrieval of those instructions by processor 502.

Data in the data caches may be copies of data in memory 504 or database 506 for instructions executing at processor 502 to operate on; the results of previous instructions executed at processor 502 for access by subsequent instructions executing at processor 502 or for writing to memory 504 or database 506; or other suitable data. The data caches may speed up read or write operations by processor 502. The TLBs may speed up virtual-address translation for processor 502. In certain embodiments, processor 502 may include one or more internal registers for data, instructions, or addresses. This disclosure contemplates processor 502 including any suitable number of any suitable internal registers, where appropriate. Where appropriate, processor 502 may include one or more arithmetic logic units (ALUs); be a multi-core processor; or include one or more processors 502. Although this disclosure describes and illustrates a particular processor, this disclosure contemplates any suitable processor.

In certain embodiments, memory 504 includes main memory for storing instructions for processor 502 to execute or data for processor 502 to operate on. As an example, and not by way of limitation, the one or more computing device(s) 500 may load instructions from database 506 or another source (such as, for example, another one or more computing device(s) 500) to memory 504. Processor 502 may then load the instructions from memory 504 to an internal register or internal cache. To execute the instructions, processor 502 may retrieve the instructions from the internal register or internal cache and decode them. During or after execution of the instructions, processor 502 may write one or more results (which may be intermediate or final results) to the internal register or internal cache. Processor 502 may then write one or more of those results to memory 504.

In certain embodiments, processor 502 executes only instructions in one or more internal registers, internal caches, or memory 504 (as opposed to database 506 or elsewhere) and operates only on data in one or more internal registers, internal caches, or memory 504 (as opposed to database 506 or elsewhere). One or more memory buses (which may each include an address bus and a data bus) may couple processor 502 to memory 504. Bus 512 may include one or more memory buses, as described below. In certain embodiments, one or more memory management units (MMUs) reside between processor 502 and memory 504 and facilitate accesses to memory 504 requested by processor 502. In certain embodiments, memory 504 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 504 may include one or more memory devices 504, where appropriate. Although this disclosure describes and illustrates particular memory, this disclosure contemplates any suitable memory.

In certain embodiments, database 506 includes mass storage for data or instructions. As an example, and not by way of limitation, database 506 may include a hard disk drive (HDD), a floppy disk drive, flash memory, an optical disc, a magneto-optical disc, magnetic tape, or a Universal Serial Bus (USB) drive, or a combination of two or more of these. Database 506 may include removable or non-removable (or fixed) media, where appropriate. Database 506 may be internal or external to the one or more computing device(s) 500, where appropriate. In certain embodiments, database 506 is non-volatile, solid-state memory. In certain embodiments, database 506 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), flash memory, or a combination of two or more of these. This disclosure contemplates mass database 506 taking any suitable physical form. Database 506 may include one or more storage control units facilitating communication between processor 502 and database 506, where appropriate. Where appropriate, database 506 may include one or more databases 506. Although this disclosure describes and illustrates particular storage, this disclosure contemplates any suitable storage.

In certain embodiments, I/O interface 508 includes hardware, software, or both, providing one or more interfaces for communication between the one or more computing device(s) 500 and one or more I/O devices. The one or more computing device(s) 500 may include one or more of these I/O devices, where appropriate. One or more of these I/O devices may enable communication between a person and the one or more computing device(s) 500. As an example, and not by way of limitation, an I/O device may include a keyboard, keypad, microphone, monitor, mouse, printer, scanner, speaker, still camera, stylus, tablet, touch screen, trackball, video camera, another suitable I/O device, or a combination of two or more of these. An I/O device may include one or more sensors. This disclosure contemplates any suitable I/O devices and any suitable I/O interfaces 508 for them. Where appropriate, I/O interface 508 may include one or more device or software drivers enabling processor 502 to drive one or more of these I/O devices. I/O interface 508 may include one or more I/O interfaces 508, where appropriate. Although this disclosure describes and illustrates a particular I/O interface, this disclosure contemplates any suitable I/O interface.

In certain embodiments, communication interface 510 includes hardware, software, or both providing one or more interfaces for communication (such as, for example, packet-based communication) between the one or more computing device(s) 500 and one or more other computing device(s) 500 or one or more networks. As an example, and not by way of limitation, communication interface 510 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 510 for it.

As an example, and not by way of limitation, the one or more computing device(s) 500 may communicate with an ad hoc network, a personal area network (PAN), a local area network (LAN), a wide area network (WAN), a metropolitan area network (MAN), one or more portions of the Internet, or a combination of two or more of these. One or more portions of one or more of these networks may be wired or wireless. As an example, the one or more computing device(s) 500 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), other suitable wireless network, or a combination of two or more of these. The one or more computing device(s) 500 may include any suitable communication interface 510 for any of these networks, where appropriate. Communication interface 510 may include one or more communication interfaces 510, where appropriate. Although this disclosure describes and illustrates a particular communication interface, this disclosure contemplates any suitable communication interface.

In certain embodiments, bus 512 includes hardware, software, or both coupling components of the one or more computing device(s) 500 to each other. As an example, and not by way of limitation, bus 512 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, another suitable bus, or a combination of two or more of these. Bus 512 may include one or more buses 512, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

FIG. 6 illustrates a diagram 600 of an example artificial intelligence (AI) architecture 602 (which may be included as part of the one or more computing device(s) 500 as discussed above with respect to FIG. 5) that may be utilized to generate a prediction of a molecular binding property of one or more target proteins as part of a streamlined process of protein purification for identifying target proteins (e.g., antibodies) and accelerating the selection process of therapeutic antibody candidates or other immunotherapy candidates by way of early identification of the most promising therapeutic antibody candidates, in accordance with the disclosed embodiments. In certain embodiments, the AI architecture 602 may be implemented utilizing, for example, one or more processing devices that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), and/or other processing device(s) that may be suitable for processing various molecular data and making one or more decisions based thereon), software (e.g., instructions running/executing on one or more processing devices), firmware (e.g., microcode), or some combination thereof.

In certain embodiments, as depicted by FIG. 6, the AI architecture 602 may include machine learning (ML) algorithms and functions 604, natural language processing (NLP) algorithms and functions 606, expert systems 608, computer-based vision algorithms and functions 610, speech recognition algorithms and functions 612, planning algorithms and functions 614, and robotics algorithms and functions 616. In certain embodiments, the ML algorithms and functions 604 may include any statistics-based algorithms that may be suitable for finding patterns across large amounts of data (e.g., “Big Data” such as genomics data, proteomics data, metabolomics data, metagenomics data, transcriptomics data, or other omics data). For example, in certain embodiments, the ML algorithms and functions 604 may include deep learning algorithms 618, supervised learning algorithms 620, and unsupervised learning algorithms 622.

In certain embodiments, the deep learning algorithms 618 may include any artificial neural networks (ANNs) that may be utilized to learn deep levels of representations and abstractions from large amounts of data. For example, the deep learning algorithms 618 may include ANNs, such as a perceptron, a multilayer perceptron (MLP), an autoencoder (AE), a convolution neural network (CNN), a recurrent neural network (RNN), long short term memory (LSTM), a grated recurrent unit (GRU), a restricted Boltzmann Machine (RBM), a deep belief network (DBN), a bidirectional recurrent deep neural network (BRDNN), a generative adversarial network (GAN), and deep Q-networks, a neural autoregressive distribution estimation (NADE), an adversarial network (AN), attentional models (AM), a spiking neural network (SNN), deep reinforcement learning, and so forth.

In certain embodiments, the supervised learning algorithms 620 may include any algorithms that may be utilized to apply, for example, what has been learned in the past to new data using labeled examples for predicting future events. For example, starting from the analysis of a known training data set, the supervised learning algorithms 620 may produce an inferred function to make predictions about the output values. The supervised learning algorithms 500 may also compare its output with the correct and intended output and find errors in order to modify the supervised learning algorithms 620 accordingly. On the other hand, the unsupervised learning algorithms 622 may include any algorithms that may applied, for example, when the data used to train the unsupervised learning algorithms 622 are neither classified nor labeled. For example, the unsupervised learning algorithms 622 may study and analyze how systems may infer a function to describe a hidden structure from unlabeled data.

In certain embodiments, the NLP algorithms and functions 606 may include any algorithms or functions that may be suitable for automatically manipulating natural language, such as speech and/or text. For example, in some embodiments, the NLP algorithms and functions 606 may include content extraction algorithms or functions 624, classification algorithms or functions 626, machine translation algorithms or functions 628, question answering (QA) algorithms or functions 630, and text generation algorithms or functions 632. In certain embodiments, the content extraction algorithms or functions 624 may include a means for extracting text or images from electronic documents (e.g., webpages, text editor documents, and so forth) to be utilized, for example, in other applications.

In certain embodiments, the classification algorithms or functions 626 may include any algorithms that may utilize a supervised learning model (e.g., logistic regression, naïve Bayes, stochastic gradient descent (SGD), k-nearest neighbors, decision trees, random forests, support vector machine (SVM), and so forth) to learn from the data input to the supervised learning model and to make new observations or classifications based thereon. The machine translation algorithms or functions 628 may include any algorithms or functions that may be suitable for automatically converting source text in one language, for example, into text in another language. The QA algorithms or functions 630 may include any algorithms or functions that may be suitable for automatically answering questions posed by humans in, for example, a natural language, such as that performed by voice-controlled personal assistant devices. The text generation algorithms or functions 632 may include any algorithms or functions that may be suitable for automatically generating natural language texts.

In certain embodiments, the expert systems 608 may include any algorithms or functions that may be suitable for simulating the judgment and behavior of a human or an organization that has expert knowledge and experience in a particular field (e.g., stock trading, medicine, sports statistics, and so forth). The computer-based vision algorithms and functions 610 may include any algorithms or functions that may be suitable for automatically extracting information from images (e.g., photo images, video images). For example, the computer-based vision algorithms and functions 610 may include image recognition algorithms 634 and machine vision algorithms 636. The image recognition algorithms 634 may include any algorithms that may be suitable for automatically identifying and/or classifying objects, places, people, and so forth that may be included in, for example, one or more image frames or other displayed data. The machine vision algorithms 636 may include any algorithms that may be suitable for allowing computers to “see”, or, for example, to rely on image sensors cameras with specialized optics to acquire images for processing, analyzing, and/or measuring various data characteristics for decision making purposes.

In certain embodiments, the speech recognition algorithms and functions 612 may include any algorithms or functions that may be suitable for recognizing and translating spoken language into text, such as through automatic speech recognition (ASR), computer speech recognition, speech-to-text (STT) 638, or text-to-speech (TTS) 640 in order for the computing to communicate via speech with one or more users, for example. In certain embodiments, the planning algorithms and functions 614 may include any algorithms or functions that may be suitable for generating a sequence of actions, in which each action may include its own set of preconditions to be satisfied before performing the action. Examples of AI planning may include classical planning, reduction to other problems, temporal planning, probabilistic planning, preference-based planning, conditional planning, and so forth. Lastly, the robotics algorithms and functions 616 may include any algorithms, functions, or systems that may enable one or more devices to replicate human behavior through, for example, motions, gestures, performance tasks, decision-making, emotions, and so forth.

Described herein include processes associated with predicting a molecular binding property of one or more proteins, as described above. This may include importing amino acid sequences of proteins and generating a molecular descriptor matrix based on the amino acid sequences. Protein molecules are formed of amino acid sequences. An amino acid sequence may be represented by a string of characters (e.g., a string of letters). In one or more examples, the amino acid sequences may be input to a machine learning model (e.g., a neural network) to generate the molecular descriptor matrix. In one or more examples, the machine learning model may be pre-trained using amino acid sequences. For example, the machine learning model may comprise a protein language model. In another example, the machine learning model may be pre-trained in an unsupervised manner. In some embodiments, the machine learning model may be configured to generate structure-based descriptors representing the sequences used to generate a protein structure.

The molecular feature matrix that is generated may be used to predict a molecular binding property of the corresponding protein. In some embodiments, the molecular descriptor matrix may be a multi-dimensional matrix (i.e., a tensor) comprised of a plurality of feature vectors representing the descriptors for each amino acid in the sequence of each protein. To determine the predicted molecular binding property, in one or more examples, the dimensions of the multi-dimensional molecular descriptor matrix (e.g., a descriptor tensor) may be reduced. In some embodiments, the multi-dimensional molecular descriptors matrix may (with per-amino acid feature vectors for each molecule) be reduced to a 2-dimensional molecular feature matrix (with molecular feature vectors for each molecule) by averaging the feature vectors across all amino acids in each molecule. In some embodiments, a feature dimensionality reduction technique used to reduce the number of feature vectors of the molecular descriptor matrix may include in particular by removing redundant feature vectors subsequent to the averaging. For instance, because some feature vectors (and/or the features included therein) may be highly correlated, a single representative feature vector may be identified to represent the collection of highly-correlated feature vectors. In some embodiments, a clustering technique (e.g., a hierarchical/agglomerative clustering technique) may be used to identify feature vectors that are similar (e.g., whose corresponding embeddings are less than a threshold distance away from one another in an embedding space). From the identified feature vectors, one or more representative feature vector may be selected from each cluster of similar feature vectors as being “representative” of that cluster.

As mentioned above, the representative feature vectors may be input to a machine learning model to obtain the prediction of the molecular binding property of the proteins. These proteins may be proteins of interest for potential drug discovery assays. The machine learning model may be trained to receive, as input, one or more representative feature vector describing one or more proteins and output the prediction of the molecular binding property of the proteins based on the representative feature vectors.

In some embodiments, the machine learning model may be trained by aligning the molecular descriptors (from a training molecular descriptor matrix generated by machine learning model 301 of FIG. 3A based on the imported amino acid sequences of one or more empirically-evaluated proteins) and predetermined batch binding data associated with the empirically-evaluated proteins. After being aligned, a supervised regression may be performed to train the machine learning model. In one or more examples, the regressor used may comprise a bagged decision tree, a bagged linear model, a non-bagged linear model, a random forest, a linear forest, or another type of regressor, or combination thereof.

In some embodiments, part of the training step comprises optimizing a set of hyper-parameters of the machine learning model. In one or more examples, the hyper-parameters may include regularization parameters, a number of estimators, a maximum tree depth, and the like. In one or more examples, the pipelines (e.g., feature-dimensionality reduction model 307A, feature selection model 309A, 309B, and regression model 311A, 311B) may be optimized jointly. The feature-dimensionality reduction model may be configured to use correlation clustering, recursive feature elimination, and/or other techniques to reduce a number of feature vectors of the molecular descriptor matrix.

In some embodiments, the training step may also include a cross-validation step where an optimized set of learnable parameters of the machine learning model are identified and then a cross-validation test is performed iteratively until the optimized set of learnable parameters are determined. For example, if the machine learning model included a decision tree structure (e.g., a random forest), the number of learnable parameters may include the number of trees and/or a depth of the trees. The optimized set of learnable parameters are selected such that they optimize the performance of the machine learning model. Using the optimized set of learnable parameters, the machine learning model may be trained to generate predictions of molecular binding properties of new amino acid sequences that are not part of the training sets.

In some embodiments, one or more additional steps may be performed to predict a molecular binding property of one or more protein molecules based on amino acid sequences. One of the goals of the disclosed techniques comprises predicting a property of a molecule to-be-assessed. In particular, how well a protein molecule binds to a resin provides valuable clinical information and/or valuable manufacturing process developability information that can be used in the development of new therapeutics. The foregoing describes an additional/alternative set of steps to the aforementioned steps that can be performed to predict the molecular binding properties based on amino acid sequences.

The techniques described herein effectuate many technical advantages. For example, as illustrated with respect to FIG. 1, testing binding properties of molecules, such as for drug discovery purposes, is a complex and time-consuming process. For example, the experimental duration of experimental example 102 of FIG. 1 for performing one or more protein purification processes may span a number of weeks. On the other hand, the execution time for the computational model-based example 104 (e.g., the machine learning models described herein) for performing one or more protein purification processes may be only minutes. Therefore, experimental example 102 describes a non-ideal process to test every potential molecule. The machine learning models described herein can reduce the amount of time expended on testing by increasing the number of molecules that can be screened in a given amount of time, or that be screened by a given researcher is a goal of model. As another example, molecular descriptor matrices can be generated using various existing protein language models (e.g., molecular descriptors 120 of FIG. 1). Thus, existing techniques can be harnessed to generate the machine learning models' inputs, thereby reducing the amount of additional data that needs to be collected and reducing the amount of additional model training needed. As still yet another example, the machine learning models described herein can be trained using less data while maintaining or increasing the models' accuracy. For instance, instead of inputting the molecular descriptor matrix into the machine learning models (which may include very large quantities of descriptors), the molecular descriptor matrix can be reduced to determining (and use as input to the machine learning models) the most-predictive feature vectors. This descriptor reduction process can further optimize the training processes for the machine learning models. For example, each training molecular descriptor matrix may be reduced by determining the most-predictive feature vectors, and the model may be trained based on the most-predictive feature vectors.

FIG. 7 illustrates another high-level workflow diagram 700 for performing feature generation 202, feature dimensionality reduction 204, feature filtering 206, recursive model-based feature elimination 207, and regression model optimization 208, in accordance with various embodiments. The descriptions of feature generation 202, feature dimensionality reduction 204, feature filtering 206, and regression model optimization 208 may apply equally here.

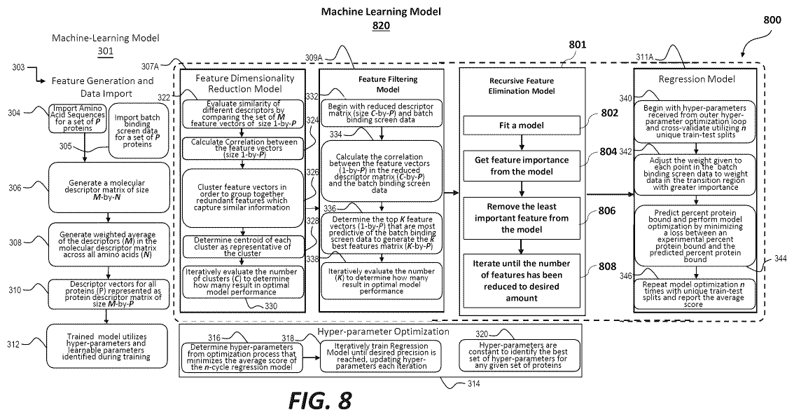

However, differing from diagram 200, diagram 700 may further include recursive model-based feature elimination 207. Recursive-model based feature elimination 207 may include an additional model for further reducing the number of features in the feature set. In particular, recursive model-based feature elimination 207 may assist in preventing or reducing the likelihood of overfitting. As an example, with reference to FIG. 8, recursive model-based feature elimination 207 may implement a machine learning model 820 of FIG. 8. FIG. 8 include similar components as that of FIG. 3A, and similar labels are used to refer to those components. For example, workflow 800 may include model 301 and machine learning model 820. Machine learning model 820 may include feature dimensionality reduction model 307A, feature filtering model 309A, recursive feature elimination model 801, and regression model 311A. Workflow 800 may follow a similar path as that of workflow 300A, with the exception that the most-predictive feature vectors may include those that have been reduced via recursive feature elimination model 801. As an example, determining the one or more most-predictive feature vectors may further comprise implementing recursive feature elimination model 801 to further reduce the number of feature vectors. In particular, some embodiments include a number of feature vectors included in the further reduced number of feature vectors being equal to or less than the number of training items.

In some embodiments, recursive feature elimination model 801, at functional block 802, may be configured to fit a model to the representative feature vectors. The model may be a regression model, for example. At functional block 804, a feature importance score may be calculated based on the fit model. The feature importance score may indicate an importance of each representative feature vector. At functional block 806, one or more feature vectors of the representative feature vectors may be removed based on the feature importance score of each of the representative feature vectors to obtain a subset of representative feature vectors. For example, a least-important feature or feature vector may be removed from the representative feature vectors. In one or more examples, the most-predictive feature vectors may comprise one or more feature vectors from the subset of representative feature vectors. At functional block 808, recursive feature elimination model 801 may iteratively perform blocks 802-806 until a number of feature vectors included in the subset satisfies a feature quantity criterion. For example, the feature quantity criterion being satisfied comprises the number of feature vectors included in the subset of representative feature vectors being less than or equal to a threshold number of feature vectors. In some examples, the threshold number of feature vectors may include a same or similar number of features from the training data used to train machine learning model 820. In some examples, the number of feature vectors included in the subset of representative feature vectors may include one of the set of hyper-parameters. In some examples, the number of feature vector clusters included in the plurality of feature vector clusters comprises one of the set of hyper-parameters.

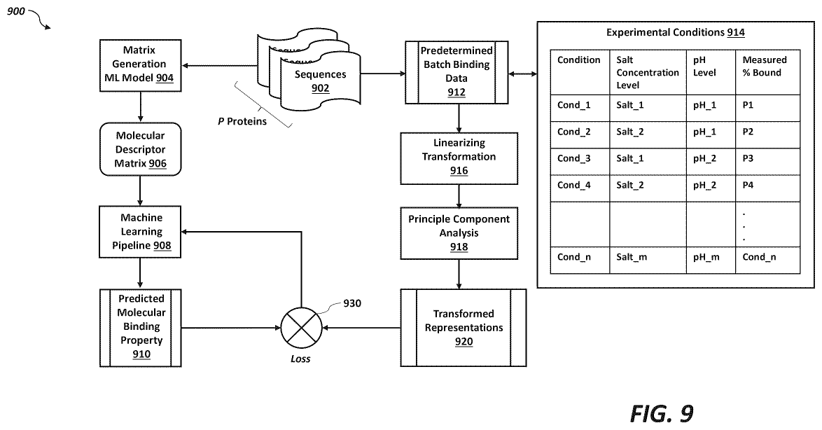

FIG. 9 illustrates a process for training a machine learning model to predict a molecular binding property, in accordance with various embodiments. As an example, as compared to the processes described above with respect to FIGS. 3A-4, process 900 of FIG. 9, as described herein, may organize the data used to train a regression model (e.g., at step 930) in a different manner. As an example, in the previously-described processes, for each empirically-evaluated protein, the data used to train the machine learning model(s) (e.g., machine learning pipeline 908) include a predefined quantity of experimental conditions. The experimental conditions may specify a molecular binding property of a protein for a given set of experimental conditions. For example, the data may comprise a measured molecular binding level of a protein at a first salt concentration and a first pH level, a measured molecular binding level of the protein at a second salt concentration and the first pH level, a measured molecular binding level of the protein at the first salt concentration and a second pH level, and the like. In one or more examples, the predefined quantity of experimental conditions for the predetermined batch binding may include 12 or more experimental conditions (e.g., 4 salt concentrations, 3 pH levels), 24 or more experimental conditions (e.g., 6 salt concentrations, 4 pH levels), and the like. The trained machine learning model, as described above, may use experimental conditions (e.g., pH levels and salt concentrations) as inputs in addition to the molecular descriptor matrix to predict a molecular binding property of the one or more proteins.

In some embodiments, the experimental conditions may not need to be input to the machine learning model and instead a predicted molecular binding property may be determined for a continuum of experimental conditions. To do so, however, the training data and training process may be adjusted, as illustrated in FIG. 9.

FIG. 9 illustrates a workflow diagram of a process 900 for optimizing hyper-parameters and learnable parameters of a machine learning model for performing one or more computational model-based protein purification processes, in accordance with various embodiments. Process 900 differs from that described above with respect to FIGS. 3A-4 in that a transformed representation of a molecular binding property of the training empirically-evaluated proteins may be used to train the machine learning model. The trained machine learning model may output a value corresponding to the transformed representation of the molecular binding property which in turn can be used to predict all binding conditions for all experimental conditions for a given protein molecule. Therefore, the amount of training data needed to train the machine learning model may be reduced from N empirically-derived binding measures for N different experimental conditions (e.g., salt concentration levels and PH levels) to a single transformed binding measure that can be used to resolve the N empirically-derived binding measures.

In process 900, sequence data 902 corresponding to one or more amino acid sequences of proteins P may be provided to a matrix generation machine learning (ML) model 904. In some embodiments, machine learning model 904 may be the same or similar to machine learning model 301 of FIG. 3A, and the previous description may apply. In some embodiments, matrix generation ML model 904 may be trained to generate a molecular descriptor matrix 906 from sequence data 902 representing the amino acid sequences of the P proteins. Matrix generation ML model 904 may comprise a neural network, which may generate features X structed as molecular descriptor matrix 906. Molecular descriptor matrix 906 may be the same or similar to the molecular descriptor matrix generated at functional block 306 of FIG. 3A. In one or more examples, molecular descriptor matrix 906 may include 100 or more features, 500 or more features, 1,000 or more features, 2,000 or more features, 10,000 or more features, or other amounts of features. The features of molecular descriptor matrix 906 may then be analyzed to determine which (if any) correlate with a molecular binding property of the corresponding protein molecule.

Molecular descriptor matrix 906 may have dimensions of a number of molecules M by a number of descriptors (e.g., features) N. For a given molecule, the amino acid sequence can be represented using a string of characters (e.g., the alphabet) that form the proteins being tested.

In some embodiments, sequences 902 may also be analyzed experimentally. The experiments may produce empirically-derived protein binding data 912. Empirically-derived protein binding data 912 may comprise molecular binding property values for a set of experimental conditions 914. For example, empirically-derived protein binding data 912 may indicate that for a given sequence (e.g., Sequence A) and a first experimental condition (e.g., a first salt concentration level and a first pH level), the molecular binding property is Y1. Similarly, empirically-derived protein binding data 912 may indicate that for the sequence (e.g., Sequence A) and a second experimental condition (e.g., a second salt concentration level and the first pH level), the molecular binding property is Y2. Still further, empirically-derived protein binding data 912 may indicate that for the sequence (e.g., Sequence A) and a third experimental condition (e.g., the first salt concentration level and a second pH level), the molecular binding property is Y3. In some embodiments, predetermined batch binding data may be formulated as with the molecules as rows and experimental conditions 914 as columns.

Process 900 may be configured to train a machine learning model (e.g., machine learning model 820) to predict a molecular binding property of a protein for a set of experimental conditions. Testing binding properties of molecules, such as for drug discovery purposes, is a complex and time-consuming process (e.g., takes 2-6 weeks to grow molecule, purify, and then test, so it can take several weeks to fully evaluate each molecule). It is not ideal to test every potential molecule. Therefore, increasing the number of molecules that can be screened in a given amount of time, or that be screened by a given researcher is a goal of the model. Another goal is increasing the number of molecules that can be screened without incurring the timeline delays or additional experimental burden.

Process 900 may be trained using a small number of training examples (e.g., few molecules) and a large number of descriptors (e.g., 100 or more features, 500 or more features, 1,000 or more features, 2,000 or more features, 10,000 or more features, etc.). Process 900 may sort the descriptors in a systematic way to train machine learning model to predict molecular binding property 910. Additionally, process 900 may leverage the descriptors which have a relationship to one or more physical attributes of the protein. Machine learning pipeline 908 may thereby be configured to find the descriptors (e.g., features) that best predict the molecular binding property of a protein based on the molecular descriptor matrix. The ML model may then try and determine which descriptors are the most predictive.

In an illustrative example, predetermined batch binding data 912 comprises empirically-measured binding properties of each analyzed protein for the set of experimental conditions. In some embodiments, process 900 may include a set of performing, for example using computing system 500 of FIG. 5, a linearizing transformation 916 to the empirically-measured binding properties. For example, the empirically-measured binding properties may comprise percent-bound measures (e.g., a protein is Y % bound to a resin). Process 900 may transform the percent-bound empirically-measured binding properties stored in predetermined batch binding data 912 into a linearized or pseudo-linear representation of the that empirically-measured binding property. For example, a logit transformation operation may be performed.

The logit transformation includes calculating the log of the ratio of the bound/not-bound protein concentrations. As a result of this transformation, the bounds transform from 0.0-1.0 (i.e., 0% bound to 100% bound) to negative infinity to positive infinity (in log (Kₚ) space). With the data linearized, linear models, such as PCA models, which converge better, can be used.

In some embodiments, process 900 may include applying one or more dimensionality reduction technique (e.g., a principal component analysis (PCA) 918) to the linear representations of the empirically-measured binding properties of each analyzed protein. PCA 918 may be configured to derive a first, second, and the like, principal component (PC) of the linearizing transformation (e.g., logit transform) of their empirically-measured binding properties. The performed PCA 918 may represent the linear representations of the empirically-measured protein binding properties to a more succinct representation. In particular, the number of experimental conditions C defines a number of data points in predetermined batch binding data 912. PCA 918 may reduce the number of data points from C to less than or equal to C. For example, if 24 experimental conditions were used to obtain predetermined batch binding data 912, the PCA can allow predetermined batch binding data 912 as less than (or equal to) 24 data points. PCA 918 may be configured to output transformed representations 920 representing the transformed versions of the empirically-measured molecular binding property. The number of molecules that are tested may be 1 or more, 5 or more, 10 or more, 20 or more, 50 or more, or other values.

The PCA model can decompose the data (e.g., predetermined batch binding data 912) into a set of lower-dimensionality vectors. For example, for 24 experimental conditions (e.g., 24 experimental data points), the PCA model can identify the first eigenvector of the data, which may capture a plurality of the variance of the data set. Thus, PCA enables a lower dimensional projection to be used to describe the behavior of the binding data. In one or more examples, if an average binding efficiency of a molecule is to be predicted, the PCA provides a more representative and valuable result than any of the experimental conditions individually. Additionally, PCA's ability to succinctly (in a low-dimensional representation) summarize trends in noisy multidimensional data can be useful to scientists. Persons of ordinary skill in the art will recognize that any number of principal components can be identified by PCA 918 including, but not limited to, a first principal component and/or a second principal component.

In some embodiments, predicted molecular binding property 910 may be compared to transformed representations 920 of the empirically-measured molecular binding property. In one or more examples, a cross-validation loss may be calculated to determine how well machine learning model 908 predicted the empirically-measured molecular binding property of a given protein. In particular, the prediction indicates how well machine learning pipeline 908 predicts a transformed representation of the empirically-measured molecular binding property.

In some embodiments, at 930, a cross-validation loss may be computed. As described previously, one or more examples may use a k-fold cross-validation technique. Additionally, or alternatively, at 930, a stratified k-fold cross-validation may be computed. The stratified k-fold cross-validation comprises taking the molecules of the training set and ranking them into bins based on their molecular binding property. For example, the bins may comprise a first bin corresponding to weakly-binding proteins, a second bin corresponding to moderately-binding proteins, a third bin corresponding to tightly-binding proteins, and the like. The stratified k-fold cross-validation may then evaluate a performance of machine learning pipeline 908 by selecting a representative subset, such as an even number of representative of each of the binned proteins.

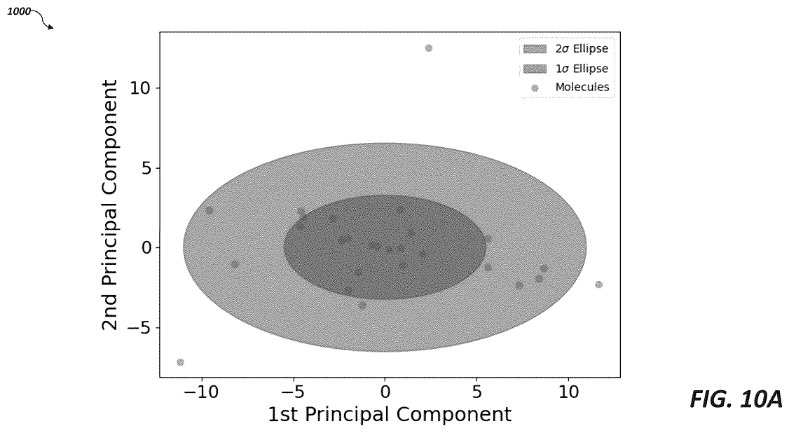

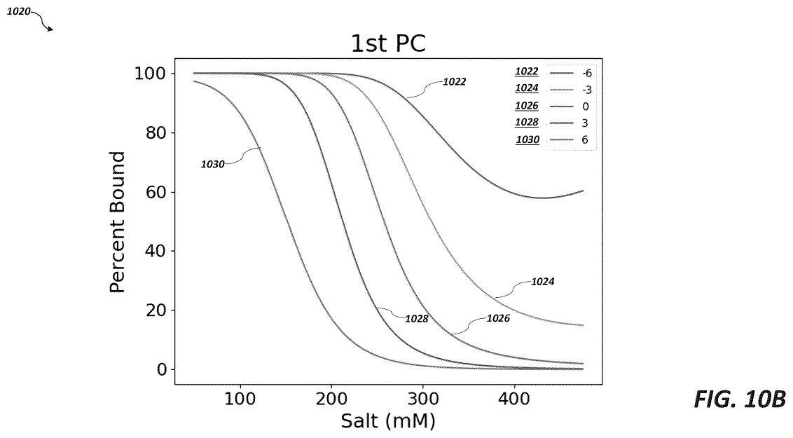

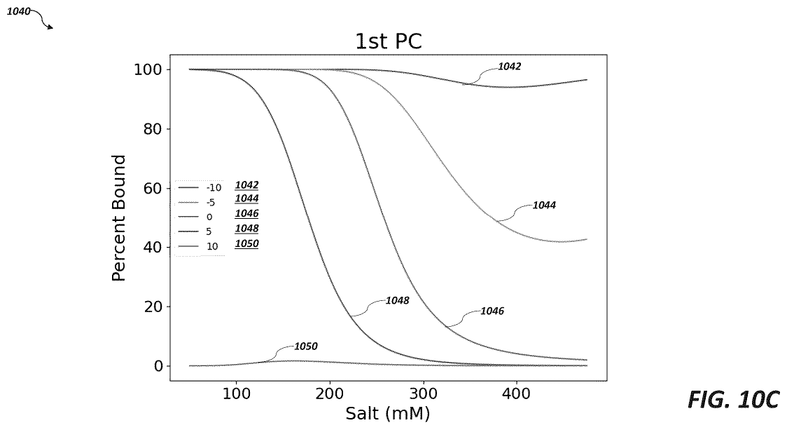

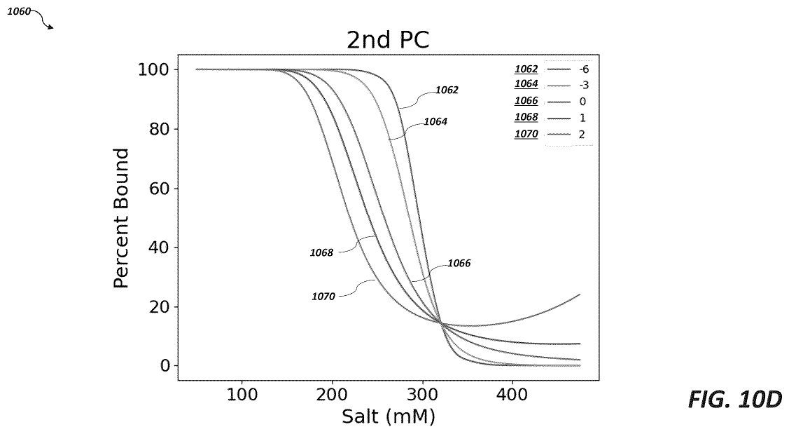

FIGS. 10A-10D illustrate example plots illustrating how a principal component analysis can be used to predict a molecular binding property, in accordance with various embodiments. FIG. 10A, for example, illustrates a plot 1000 of a principal component analysis result of a set of molecules. In plot 1000, the X-axis corresponds to a 1^{st} principal component value of each molecule of the set and the Y-axis corresponds to a 2^{nd} principal component of each molecule. The red oval and the green oval represent a first and second standard deviation from a centroid of the cluster of data points. As can be seen in plot 1000, the molecules (e.g., data points) are fairly well-distributed about the x-axis.

FIG. 10B illustrates a plot 1020 of isotherm curves for a given molecule for various values of a first principal component, in accordance with various embodiments. In plot 1020, the x-axis represents a salt concentration level used during a corresponding experiment to determine a protein binding property of a molecule and the y-axis represents a protein binding level. Isotherm curves 1022-1030 correspond to different principal component (PC) values. For example, isotherm curve 1022 represents how a binding of a protein changes as a salt concentration level is varied for a first PC (e.g., PC=−6). Isotherm curve 1024 represents how a binding of the protein changes as the salt concentration level is varied for a second PC (e.g., PC=−3). Isotherm curve 1026 represents how a binding of the protein changes as the salt concentration level is varied for a third PC (e.g., PC=0). Isotherm curve 1028 represents how a binding of the protein changes as the salt concentration level is varied for a fourth PC (e.g., PC=+3). Isotherm curve 1030 represents how a binding of the protein changes as the salt concentration level is varied for a fifth PC (e.g., PC=+6).

Isotherm curves 1022-1030 of plot 1020 may be computed using a fixed pH level. As seen from plot 1020, as the value of the first PC increases (e.g.,-6 in curve 1022) to very large (e.g., +6 in curve 1030), the binding behavior changes. In the example of plot 1020, the percent bound is approximately 100% for low salt concentrations and approximately 0% for high salt concentration values.

When manufacturing a protein, one or more protein purification steps may be performed to filter out molecules that are not a protein of interest. The protein purification step include causing or otherwise facilitating the protein of interest to bind to a resin (e.g., a chromatography column). Ideally, the resin will bind all of the proteins of interest. Then, to remove the proteins from the resin, a wash may be applied to deposit the proteins of interest into a solution. The wash may include salt at a particular salt concentration level (and/or PH level). The salt concentration level may influence whether the protein un-binds from the resin. For example, at lower salt concentration levels, a protein may remain bound to a resin, whereas higher salt concentration levels may cause the protein to detach from the resin. Once removed, assays or other studies may be performed to the solution/protein.

In general, for all resins, molecules that are fully or mostly bound across an entire design space (typical range of pH and salt for which the protein is stable) may not be compatible due to inability to elute the protein resulting in low yield. For resins that are typically operated in a bind and elute mode, molecules that are completely or mostly unbound across the entire design space may not be compatible due to inability to bind the protein. It is therefore desirable to have a protein that expresses a variability in its binding—the binding should transition from a bound state (e.g., greater than 90% bound) to an unbound state (e.g., less than 10% bound). The first principal component, as illustrated by FIGS. 10A-10B, describes the transition of the protein from a bound to unbound state (e.g., as seen by isotherm curves 822-830) using a single value (e.g., the principal component) instead of the set of experimental conditions (e.g., 24 salt/pH combinations).

In some embodiments, the first principal component, as illustrated in plot 1020 of FIG. 10B, can visually describe the average binding, as a percent bound. For example, as seen by isotherm curve 1022, for a first principal component of −6, the protein may be tightly bound to the resin. Isotherm curve 1022 may be flagged as problematic because, regardless of the salt concentration level, for the particular pH level and first principal component value, the protein under analysis is unlikely to unbind from the resin.

Looking at plot 1040 of FIG. 10C, a different set of principal component values may be analyzed. The protein under analysis and the pH level used in the example of plot 1040 may be similar to that of plot 1020 of FIG. 10B, however this is not required. As seen in plot 1040, isotherm curves 1042-1050 illustrate how the binding percentage varies as the salt concentration level of the wash changes for different values of the first principal component. For example, isotherm curve 1042 represents how a binding of a protein changes as a salt concentration level is varied for a first PC (e.g., PC=−10). Isotherm curve 1044 represents how a binding of the protein changes as the salt concentration level is varied for a second PC (e.g., PC=−5). Isotherm curve 1046 represents how a binding of the protein changes as the salt concentration level is varied for a third PC (e.g., PC=0). Isotherm curve 1048 represents how a binding of the protein changes as the salt concentration level is varied for a fourth PC (e.g., PC=+5). Isotherm curve 1050 represents how a binding of the protein changes as the salt concentration level is varied for a fifth PC (e.g., PC=+10).

Looking at isotherm curve 1042, the percent bound of the protein does not change much as the salt concentration level is varied. Isotherm curve 1042 may then also be flagged as problematic because the protein is bound to the resin and cannot be removed. As another example, isotherm curve 1050 may have a substantially static percent bound regardless of salt concentration level. However, differing from isotherm curve 1042, the protein in this example may not be able to bind to the resin. Isotherm curve 1050 may therefore also be flagged as problematic because no purification can be performed, as all of the protein washes away. Isotherm curves 1044-1048 represent a more desirable state, where the percent bound transitions from bound to unbound as the salt concentration level is varied.

In some embodiments, predicting the first principal component can enable the percent bound to be determined for an infinite amount of salt concentrations (and/or pHs). In contrast, without use of the PCA, a percent bound prediction for all experimental conditions (e.g., points along an isotherm curve) would be needed. Thus, use of PCA to predict a first principal component vastly simplifies the process of predicting a molecular binding property of a protein without sacrificing accuracy.

In some embodiments, the PCA may output more than the first principal component. For example, the second principal component may also be determined and may be used to guide decision making steps. As an example, with reference to FIG. 10D, plot 1060 depicts isotherm curves 1062-1070 of a second principal component for a protein. Isotherm curves 1062-1070 illustrate how the percent bound of the protein changes as the salt concentration level is varied for a set of second principal component values. For example, isotherm curve 1062 represents how a binding of a protein changes as a salt concentration level is varied for a first PC value (e.g., 2^{nd} PC value=−6). Isotherm curve 1064 represents how a binding of the protein changes as the salt concentration level is varied for a second PC value (e.g., 2^{nd} PC value=−3). Isotherm curve 1066 represents how a binding of the protein changes as the salt concentration level is varied for a third PC value (e.g., 2^{nd} PC value=0). Isotherm curve 1068 represents how a binding of the protein changes as the salt concentration level is varied for a fourth PC value (e.g., 2^{nd} PC value=+1). Isotherm curve 1070 represents how a binding of the protein changes as the salt concentration level is varied for a first PC value (e.g., 2^{nd} PC value=+2). The first principal component can shift where the transition is from bound to unbound. In some examples, that transition may not even occur. The second principal component does not shift the transition as much but does changes the steepness of isotherm curves 1062-1070. For example, isotherm curve 1062 may include a 2^{nd} PC value of −6, which as illustrated is very steep as compared with isotherm curve 1070, having a 2^{nd} PC value of +2, is less steep (and does not reach a percent bound of ˜ 0%). In some embodiments, other principal components may be used to. In plot 1060 of FIG. 10D, isotherm curve 1066 may represent an “ideal” curve. In this example, the first principal component may be set at 0 while the second principal component is varied.

In some embodiments, machine learning pipeline 908 may be trained to output the first principal component, the second principal component, other principal components, or combinations thereof. Machine learning pipeline 908 may output the principal components together or serially.

In some embodiments, process 900 can reduce a number of data points needed to train machine learning model. For example, the number of principal components may be limited by the number of data points of the empirically-measured proteins. In one or more examples, the number of principal components may be less than or equal to the number of experimental conditions. For example, while the process described by FIGS. 3A-4 may require N data points for N experimental conditions, process 900 of FIG. 9 may reduce that number to 1 data point.

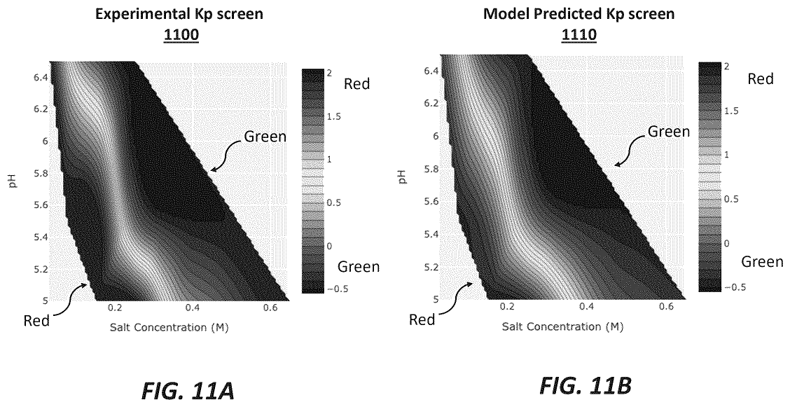

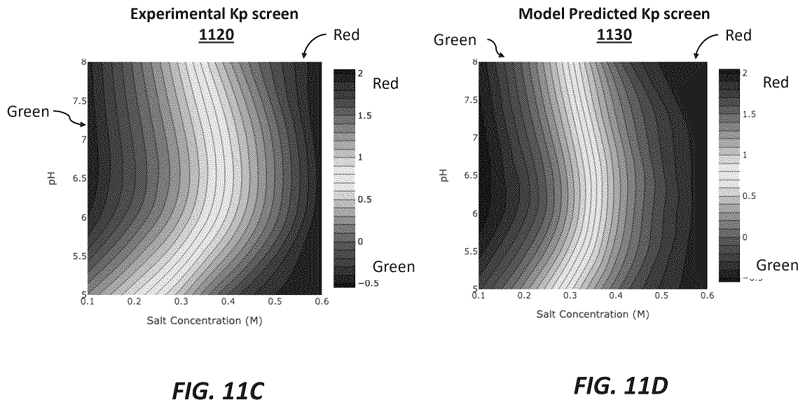

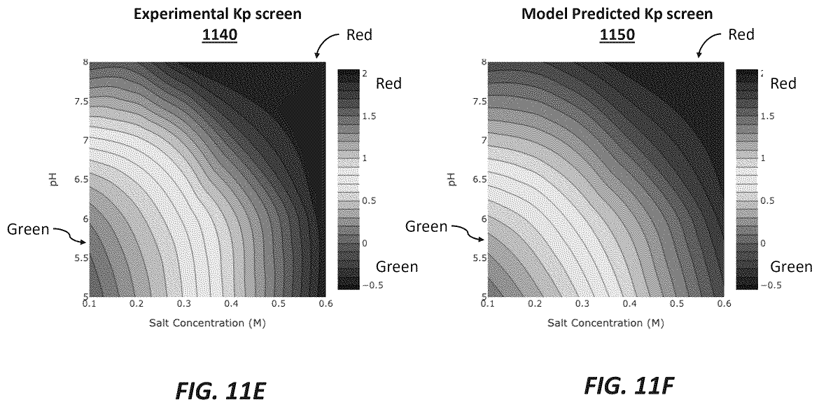

FIGS. 11A-11F illustrate example heat maps 1100-1150 illustrating a relationship between experimental conditions and experimental Kₚ values, and experimental conditions and modeled Kₚ values, respectively, in accordance with various embodiments. Heat maps 1100-1150 include a color gradient representing how tightly bound a protein is (in units of percent bound). The x-axis of maps 1100-1150 describes a salt concentration level and the y-axis represents a pH level. The portions of heat maps 1100-1150 that are “red” represent a higher log (Kₚ) value (e.g., molecular binding property) and the “green” represents a lower log (Kₚ) value. Heat map 1100-1150 may be generated based on the one or more empirically-evaluated proteins. For example, FIGS. 11A-11B may depict heat maps 1100-1110 depicting an experimental Kₚ screen and a model predicted Kₚ screen for an ion exchange resin. FIGS. 11C-11D may depict heat maps 1120-1130 depicting an experimental Kₚ screen and a model predicted Kₚ screen for a hydrophobic resin. FIGS. 11E-11F may depict heat maps 1140-1150 depicting an experimental Kₚ screen and a model predicted Kₚ screen for a mixed mode resin. As an example, at a pH level of pH=5.5, the protein of interest may be bound until the salt concentration level used reaches approximately 250 mM in the experimental data. On the other hand, the modeled data may indicate that, for a pH level of pH=5.5, the protein of interest may be bound until the salt concentration level reaches approximately 175 mM.

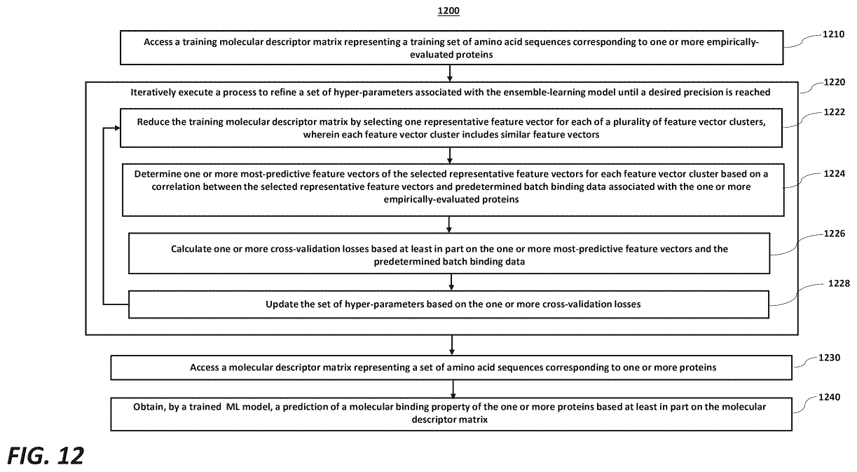

FIG. 12 illustrates a flow diagram of a method 1200 for generating a prediction of a molecular binding property of one or more target proteins as part of another streamlined process of protein purification for identifying target proteins, in accordance with various embodiments. Method 1200 may accelerate the selection process of therapeutic antibody candidates or other immunotherapy candidates by way of early identification of the most promising therapeutic antibody candidates, in accordance with the disclosed embodiments. Method 1200 may be performed utilizing one or more processing devices (e.g., computing device(s) and artificial intelligence architecture to be discussed below with respect to FIGS. 5 and 6) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other processing device(s) that may be suitable for processing genomics data, proteomics data, metabolomics data, metagenomics data, transcriptomics data, or other omics data and making one or more processors), firmware (e.g., microcode), or some combination thereof.

In some embodiments, method 1200 may begin at block 1210. Block 1210 may form part of the steps performed to train machine learning pipeline 908. At block 1210, a training molecular descriptor matrix representing a training set of amino acid sequences corresponding to one or more empirically-evaluated proteins may be accessed. For example, the training molecular matrix may be generated for proteins that have been evaluated experimentally under one or more experimental conditions (e.g., salt concentration levels, pH levels, etc.).

At block 1220, an iterative process may be executed to refine a set of hyper-parameters associated with the ensemble-learning model until a desired precision is reached. For example, the process may repeat until machine learning pipeline 908 predicts molecular binding properties with a threshold level of accuracy. Block 1220 may include a steps that are performed during each iteration of block 1220. For example, at step 1222, the training molecular descriptor matrix may be reduced by selecting one representative feature vector for each of a plurality of feature vector clusters. Each feature vector cluster may comprise similar feature vectors. For example, two feature vectors having a distance less than a threshold distance (e.g., in an embedding space) may be classified as being “similar.” The selected representative feature vector may represent all the feature vectors included within a given feature vector cluster.

At step 1224, one or more most-predictive feature vectors of the selected representative feature vectors for each feature vector cluster may be determined based on a correlation between the selected representative feature vectors and predetermined batch binding data associated with the empirically-evaluated proteins. The most-predictive feature vectors may be determined based on a principal component analysis identifying a first principal component.

Step 1226, one or more cross-validation losses may be calculated based at least in part on the most-predictive feature vectors and the predetermined batch binding data. The set of hyper-parameters of machine learning pipeline 908 may be updated based on the cross-validation losses. At step 1228, the set of hyper-parameters may be updated based on the one or more cross-validation losses.

Blocks 1210-1220 (including steps 1222-1228) may comprise a “training” portion. The result of blocks 1210-1220 may include the trained machine learning model (e.g., machine learning model 908), which can be used during inferencing. For example, at block 1230, a molecular descriptor matrix representing a set of amino acid sequences corresponding to one or more proteins may be accessed. At block 1240, a prediction of a molecular binding property of the one or more proteins may be obtained by a trained ML model based at least in part on the molecular descriptor matrix.

In some embodiments, the proteins may be proteins of interest. In one or more examples, a machine learning model (e.g., a protein language model implemented using a neural network) may be trained to receive data representing a set of amino acid sequences corresponding to the proteins and generate a molecular descriptor matrix describing the amino acid sequences. In some examples, the molecular descriptor matrix may comprise a plurality of descriptors (e.g., features). The descriptors may be structured as feature vectors.

In some embodiments, machine learning pipeline 908 may be trained to analyze the molecular descriptor matrix and perform a dimensionality reduction. The dimensionality reduction may reduce the molecular descriptor matrix by selecting a representative feature vector. The selected representative feature vector may be selected from a cluster of similar feature vectors of the molecular descriptor matrix. In one or more examples, each cluster may have a representative feature vector. The most-predictive feature vectors of the representative feature vectors may be determined. The most-predictive feature vectors may then be used to generate a predicted molecular binding property. In one or more examples, the predicted molecular binding property may represent a first principal component.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

Herein, “automatically” and its derivatives means “without human intervention,” unless expressly indicated otherwise or indicated otherwise by context.

The embodiments disclosed herein are only examples, and the scope of this disclosure is not limited to them. Embodiments according to this disclosure are in particular disclosed in the attached claims directed to a method, a storage medium, a system and a computer program product, wherein any feature mentioned in one claim category, e.g., method, may be claimed in another claim category, e.g., system, as well. The dependencies or references back in the attached claims are chosen for formal reasons only. However, any subject matter resulting from a deliberate reference back to any previous claims (in particular multiple dependencies) may be claimed as well, so that any combination of claims and the features thereof are disclosed and may be claimed regardless of the dependencies chosen in the attached claims. The subject-matter which may be claimed comprises not only the combinations of features as set out in the attached claims but also any other combination of features in the claims, wherein each feature mentioned in the claims may be combined with any other feature or combination of other features in the claims. Furthermore, any of the embodiments and features described or depicted herein may be claimed in a separate claim and/or in any combination with any embodiment or feature described or depicted herein or with any of the features of the attached claims.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates certain embodiments as providing particular advantages, certain embodiments may provide none, some, or all of these advantages.

### EXAMPLE EMBODIMENTS

Embodiments disclosed herein may include:

1. A method for predicting a molecular binding property of one or more proteins, comprising, by one or more computing devices: accessing a molecular descriptor matrix representing a set of amino acid sequences corresponding to one or more proteins; and refining a set of hyper-parameters associated with a machine learning model trained to generate a prediction of a molecular binding property of the one or more proteins, wherein refining the set of hyper-parameters comprises iteratively executing a process until a desired precision is reached, the process comprising: reducing the molecular descriptor matrix by selecting one representative feature vector for each of a plurality of feature vector clusters, wherein each feature vector cluster includes similar feature vectors; determining one or more most-predictive feature vectors of the selected representative feature vectors for each feature vector cluster based on a correlation between the selected representative feature vectors and predetermined batch binding data associated with the one or more proteins; calculating one or more cross-validation losses based at least in part on the one or more most-predictive feature vectors and the predetermined batch binding data; and updating the set of hyper-parameters based on the one or more cross-validation losses; and outputting, by the machine learning model, the prediction of the molecular binding property of the one or more proteins based at least in part on the updated set of hyper-parameters.

2. The method of embodiment 1, wherein calculating the one or more cross-validation losses further comprises: evaluating a cross-validation loss function based on the one or more most-predictive feature vectors, the predetermined batch binding data, the set of hyper-parameters, and a set of learnable parameters associated with the machine learning model; and minimizing the cross-validation loss function by varying the set of learnable parameters while the one or more most-predictive feature vectors, the predetermined batch binding data, and the set of hyper-parameters remain constant.

3. The method of embodiment 2, wherein minimizing the cross-validation loss function comprises optimizing the set of hyper-parameters, and wherein the set of hyper-parameters comprises one or more of a set of general parameters, a set of booster parameters, or a set of learning-task parameters.

4. The method of embodiment 2 or 3, wherein minimizing the cross-validation loss function comprises minimizing a loss between a prediction of a percent protein bound for the one or more proteins and an experimentally-determined percent protein bound for the one or more proteins.

5. The method of any one of embodiments 2-4, wherein the predetermined batch binding data comprises an experimentally-determined percent protein bound for one or more pH values and salt concentrations associated with the molecular binding property of the one or more proteins.

6. The method of any one of embodiments 2-5, wherein the set of learnable parameters comprises one or more weights or decision variables determined by the machine learning model based at least in part on the one or more most-predictive feature vectors and the predetermined batch binding data.

7. The method of any one of embodiments 1-6, further comprising: subsequent to refining the set of hyper-parameters: accessing a second molecular descriptor matrix representing a second set of amino acid sequences corresponding to one or more second proteins; reducing the second molecular descriptor matrix by selecting one representative feature vector for each of a second plurality of feature vector clusters of the second molecular descriptor matrix; determining one or more second most-predictive feature vectors of the selected representative feature vectors for each feature vector cluster based on a second correlation between the selected representative feature vectors and predetermined batch binding data associated with the one or more second proteins; inputting the one or more second most-predictive feature vectors into the machine learning model trained to generate a prediction of a molecular binding property of the one or more second proteins; and outputting, by the machine learning model, the prediction of the molecular binding property of the one or more second proteins based at least in part on the updated set of hyper-parameters.

8. The method of embodiment 7, wherein the prediction of the molecular binding property of the one or more second proteins comprises a prediction of a percent protein bound for the one or more second proteins.

9. The method of any one of embodiments 1-8, wherein the updated set of hyper-parameters comprises one or more of an updated set of general parameters, an updated set of booster parameters, or an updated set of learning-task parameters.

10. The method of any one of embodiments 1-9, wherein calculating the one or more cross-validation losses comprises calculating an n number of cross-validation losses, and wherein n comprises an integer from 1-n.

11. The method of any one of embodiments 1-10, wherein calculating the one or more cross-validation losses comprises determining an n number of individual train-test splits based on the one or more most-predictive feature vectors and the predetermined batch binding data, and wherein n comprises an integer from 1-n.

12. The method of any one of embodiments 1-11, wherein calculating the one or more cross-validation losses comprises calculating an n number of cross-validation losses, the method further comprising: generating the prediction of the molecular binding property of the one or more proteins based on an averaging of the n number of cross-validation losses.

13. The method of any one of embodiments 1-12, wherein the molecular descriptor matrix comprises 2″ feature vectors, and wherein n comprises a dimension of the molecular descriptor matrix.

14. The method of any one of embodiments 1-13, wherein the molecular descriptor matrix was generated by a first machine learning model distinct from the machine learning model.

15. The method of embodiment 14, wherein the first machine learning model was trained to generate the molecular descriptor matrix based on the set of amino acid sequences.

16. The method of embodiment 14 or 15, wherein the first machine learning model comprises a neural network trained to generate an M×N descriptor matrix representing the set of amino acid sequences, and wherein N comprises a number of the set of amino acid sequences and M comprises a number of nodes in an output layer of the neural network.

17. The method of any one of embodiments 1-16, wherein the machine learning model comprises one or more of a gradient boosting model, an adaptive boosting (AdaBoost) model, an eXtreme gradient boosting (XGBoost) model, a light gradient boosted machine (LightGBM) model, or a categorical boosting (CatBoost) model.

18. The method of any one of embodiments 1-17, wherein the machine learning model is further trained to generate a prediction of a molecular elution property of the one or more proteins.

19. The method of any one of embodiments 1-18, wherein the machine learning model is further trained to generate a prediction of a flow-through property of the one or more proteins.

20. The method of any one of embodiments 1-19, wherein reducing the molecular descriptor matrix comprises performing a Pearson's correlation of feature vectors of the molecular descriptor matrix to generate the plurality of feature vector clusters.

21. The method of embodiment 20, wherein the selected one representative feature vector for each of the plurality of feature vector clusters comprises a centroid feature vector for each of the plurality of feature vector clusters utilized to represent two or more of the similar feature vectors.

22. The method of any one of embodiments 1-21, wherein determining the one or more representative feature vectors of the selected representative feature vectors for each of the plurality of feature vector clusters comprises selecting a k-best matrix of feature vectors of the selected representative feature vectors for each of the plurality of feature vector clusters.

23. The method of embodiment 22, wherein the k-best matrix of feature vectors of the selected representative feature vectors is determined based on a predetermined k-best process.

24. The method of any one of embodiments 1-23, wherein the correlation between the selected representative feature vectors and the predetermined batch binding data is determined based on a maximal information coefficient (MIC) between the selected representative feature vectors and the predetermined batch binding data.

25. The method of any one of embodiments 1-24, wherein the prediction of the molecular binding property of the one or more proteins comprises a computational model-based chromatography process.

26. The method of embodiment 25, wherein the computational model-based chromatography process comprises one or more of a computational model-based affinity chromatography process, ion exchange chromatography (IEX) process, a hydrophobic interaction chromatography (HIC) process, or a mixed-mode chromatography (MMC) process.

27. The method of any one of embodiments 1-26, further comprising optimizing the machine learning model based on a Bayesian model-optimization process.

28. The method of embodiment 27, further comprising utilizing Group K-Fold cross-validation to train and evaluate the optimized machine learning model based on the one or more most-predictive feature vectors, the predetermined batch binding data, the set of hyper-parameters, and the set of learnable parameters.

29. The method of any one of embodiments 1-28, wherein the prediction of the molecular binding property of the one or more proteins comprises an identification of a target protein of the one or more proteins.

30. The method of any one of embodiments 1-29, wherein the prediction of the molecular binding property of the one or more proteins comprises a quantitative structure property relationship (QSPR) or a quantitative structure activity relationship (QSAR) modeling of the one or more proteins.

31. The method of any one of embodiments 1-30, wherein the prediction of the molecular binding property of the one or more proteins comprises a prediction of a molecular binding property for each amino acid sequence of the set of amino acid sequences corresponding to the one or more proteins.

32. A method for predicting a molecular binding property of one or more proteins, comprising, by one or more computing devices: accessing a molecular descriptor matrix representing a set of amino acid sequences corresponding to one or more proteins; and obtaining, by a machine learning model, a prediction of a molecular binding property of the one or more proteins based at least in part on the molecular descriptor matrix, wherein the machine learning model is trained by: accessing a training molecular descriptor matrix representing a training set of amino acid sequences corresponding to one or more empirically-evaluated proteins; and iteratively executing a process to refine a set of hyper-parameters associated with the machine learning model until a desired precision is reached, the process comprising: reducing the training molecular descriptor matrix by selecting one representative feature vector for each of a plurality of feature vector clusters, wherein each feature vector cluster includes similar feature vectors; determining one or more most-predictive feature vectors of the selected representative feature vectors for each feature vector cluster based on a correlation between the selected representative feature vectors and predetermined batch binding data associated with the one or more empirically-evaluated proteins; and calculating one or more cross-validation losses based at least in part on the one or more most-predictive feature vectors and the predetermined batch binding data; and updating the set of hyper-parameters based on the one or more cross-validation losses.

33. The method of embodiment 32, wherein obtaining the prediction comprises: reducing the molecular descriptor matrix by selecting one representative feature vector for each of a plurality of feature vector clusters of the molecular descriptor matrix; determining one or more most-predictive feature vectors of the selected representative feature vectors for each feature vector cluster based on a correlation between the selected representative feature vectors and predetermined batch binding data associated with the one or more proteins; inputting the one or more most-predictive feature vectors into the machine learning model to obtain the prediction of the molecular binding property of the one or more proteins.

34. The method of any one of embodiments 32-33, wherein the prediction of the molecular binding property of the one or more proteins comprises a prediction of a percent protein bound for the one or more proteins.

35. The method of any one of embodiments 32-34, wherein calculating the one or more cross-validation losses further comprises: evaluating a cross-validation loss function based on the one or more most-predictive feature vectors, the predetermined batch binding data, the set of hyper-parameters, and a set of learnable parameters associated with the machine learning model; and minimizing the cross-validation loss function by varying the set of learnable parameters while the one or more most-predictive feature vectors, the predetermined batch binding data, and the set of hyper-parameters remain constant.

36. The method of embodiment 35, wherein minimizing the cross-validation loss function comprises optimizing the set of hyper-parameters, and wherein the set of hyper-parameters comprises one or more of a set of general parameters, a set of booster parameters, or a set of learning-task parameters.

37. The method of embodiment 35 or 36, wherein minimizing the cross-validation loss function comprises minimizing a loss between a prediction of a percent protein bound for the one or more proteins and an experimentally-determined percent protein bound for the one or more proteins.

38. The method of any one of embodiments 35-37, wherein the predetermined batch binding data comprises an experimentally-determined percent protein bound for one or more pH values and salt concentrations associated with the molecular binding property of the one or more proteins.

39. The method of any one of embodiments 35-38, wherein the set of learnable parameters comprises one or more weights or decision variables determined by the machine learning model based at least in part on the one or more most-predictive feature vectors and the predetermined batch binding data.

40. The method of any one of embodiments 32-39, wherein the molecular descriptor matrix comprises a first molecular descriptor matrix representing a first set of amino acid sequences corresponding to one or more first proteins, and the prediction of the molecular binding property comprises a first prediction of a molecular binding property of the one or more first proteins, the method further comprises: accessing a second molecular descriptor matrix representing a second set of amino acid sequences corresponding to one or more second proteins; and obtaining, by the machine learning model, a second prediction of a molecular binding property of the one or more second proteins based at least in part on the second molecular descriptor matrix.

41. The method of embodiment 40, wherein the machine learning model is trained to: reduce the second molecular descriptor matrix by selecting one representative feature vector for each of a second plurality of feature vector clusters of the second molecular descriptor matrix; determine one or more second most-predictive feature vectors of the selected representative feature vectors for each feature vector cluster based on a second correlation between the selected representative feature vectors and predetermined batch binding data associated with the one or more second proteins; inputting the one or more second most-predictive feature vectors into the machine learning model trained to generate the second prediction.

42. The method of embodiment 40 or 41, wherein the second prediction of the molecular binding property of the one or more second proteins comprises a prediction of a percent protein bound for the one or more second proteins.

43. The method of any one of embodiments 32-42, wherein the updated set of hyper-parameters comprises one or more of an updated set of general parameters, an updated set of booster parameters, or an updated set of learning-task parameters.

44. The method of any one of embodiments 32-43, wherein the machine learning model used to generate the prediction of the molecular binding property of the one or more proteins comprises the updated set of hyper-parameters.

45. The method of any one of embodiments 32-44, wherein calculating the one or more cross-validation losses comprises calculating an n number of cross-validation losses, and wherein n comprises an integer from 1-n.

46. The method of any one of embodiments 32-45, wherein calculating the one or more cross-validation losses comprises determining an n number of individual train-test splits based on the one or more most-predictive feature vectors and the predetermined batch binding data, and wherein n comprises an integer from 1-n.

47. The method of any one of embodiments 32-46, wherein calculating the one or more cross-validation losses comprises calculating an n number of cross-validation losses, the method further comprising: generating the prediction of the molecular binding property of the one or more proteins based on an averaging of the n number of cross-validation losses.

48. The method of any one of embodiments 32-48, wherein the molecular descriptor matrix was generated by a first machine learning model distinct from the machine learning model.

49. The method of embodiment 48, wherein the first machine learning model was trained to generate the molecular descriptor matrix based on the set of amino acid sequences.

50. The method of embodiment 49, wherein the first machine learning model comprises a neural network trained to generate an M×N descriptor matrix representing the set of amino acid sequences.

51. The method of embodiment 49 or 50, wherein N comprises a number of the set of amino acid sequences and M comprises a number of nodes in an output layer of the neural network.

52. The method of any one of embodiments 32-51, wherein the machine learning model comprises one or more of a gradient boosting model, an adaptive boosting (AdaBoost) model, an eXtreme gradient boosting (XGBoost) model, a light gradient boosted machine (LightGBM) model, or a categorical boosting (CatBoost) model.

53. The method of any one of embodiments 32-52, wherein the machine learning model is further trained to generate a prediction of a molecular elution property of the one or more proteins.

54. The method of any one of embodiments 32-53, wherein the machine learning model is further trained to generate a prediction of a flow-through property of the one or more proteins.

55. The method of any one of embodiments 32-54, wherein reducing the molecular descriptor matrix comprises clustering the similar feature vectors into the plurality of feature vector clusters based on a correlation distance.

56. The method of embodiment 55, wherein the correlation distance is calculated using a Pearson's correlation.

57. The method of embodiment 55 or 56, wherein the selected one representative feature vector for each of the plurality of feature vector clusters comprises a centroid feature vector for each of the plurality of feature vector clusters utilized to represent two or more of the similar feature vectors.

58. The method of any one of embodiments 32-57, wherein determining the one or more predictive feature vectors of the selected representative feature vectors for each of the plurality of feature vector clusters comprises selecting a k-best matrix of feature vectors of the selected representative feature vectors for each of the plurality of feature vector clusters.

59. The method of embodiment 58, wherein the k-best matrix of feature vectors of the selected representative feature vectors is determined based on a predetermined k-best process.

60. The method of any one of embodiments 32-59, wherein the correlation between the selected representative feature vectors and the predetermined batch binding data is determined based on a maximal information coefficient (MIC) between the selected representative feature vectors and the predetermined batch binding data.

61. The method of any one of embodiments 32-60, wherein the prediction of the molecular binding property of the one or more proteins comprises a computational model-based chromatography process.

62. The method of embodiment 61, wherein the computational model-based chromatography process comprises one or more of a computational model-based affinity chromatography process, ion exchange chromatography (IEX) process, a hydrophobic interaction chromatography (HIC) process, or a mixed-mode chromatography (MMC) process.

63. The method of any one of embodiments 32-62, further comprising optimizing the machine learning model based on a Bayesian model-optimization process.

64. The method of embodiment 63, further comprising utilizing Group K-Fold cross-validation to train and evaluate the optimized machine learning model based on the one or more most-predictive feature vectors, the predetermined batch binding data, the set of hyper-parameters, and a set of learnable parameters.

65. The method of embodiment 63 or 64, further comprising utilizing stratified K-Fold cross-validation to train and evaluate the optimized machine learning model based on the one or more most-predictive feature vectors, the predetermined batch binding data, the set of hyper-parameters, and a set of learnable parameters.

66. The method of any one of embodiments 32-65, wherein the prediction of the molecular binding property of the one or more proteins comprises an identification of a target protein of the one or more proteins.

67. The method of any one of embodiments 32-66, wherein the prediction of the molecular binding property of the one or more proteins comprises a quantitative structure property relationship (QSPR) or a quantitative structure activity relationship (QSAR) modeling of the one or more proteins.

68. The method of any one of embodiments 32-67, wherein the prediction of the molecular binding property of the one or more proteins comprises a prediction of a molecular binding property for each amino acid sequence of the set of amino acid sequences corresponding to the one or more proteins.

69. The method of any one of embodiments 32-68, wherein for each of the one or more empirically-evaluated proteins, a corresponding predetermined batch binding is measured for each of a set of experimental conditions.

70. The method of embodiment 69, wherein the set of experimental conditions comprises 24 experimental conditions.

71. The method of embodiment 70, wherein the set of experimental conditions comprises a first subset of salt concentrations and a second subset of pH values.

72. The method of embodiment 70 or 71, wherein the set of experimental conditions are input to the machine learning model with the molecular descriptor matrix and the prediction of the molecular binding property of the one or more proteins comprises a prediction of a molecular binding property of the one or more proteins for each of the set of experimental conditions.

73. The method of any one of embodiments 32-72, further comprising: transforming the prediction of the molecular binding property of the one or more proteins into linear representations.

74. The method of embodiment 73, wherein a logit transformation is used to generate the linear representations.

75. The method of embodiment 73 or 74, further comprising: performing a principal component analysis (PCA) to the linear representations to obtain at least a first principal component.

76. The method of any one of embodiments 32-75, wherein the predetermined batch binding data associated with the one or more empirically-evaluated proteins comprises, for each of the one or more empirically-evaluated proteins, an experimentally-determined binding value measured for each of a set of experimental conditions.

77. The method of embodiment 76, wherein the correlation between the selected representative feature vectors and the predetermined batch binding data comprises: for each of the one or more empirically-evaluated proteins and for each of the set of experimental conditions: generating a linear representation of the experimentally-determined binding value of the empirically-evaluated protein based on a logit transformation applied to the experimentally-determined binding value of the empirically-evaluated protein; and performing a principal component analysis (PCA) to the linear representations of the experimentally-determined binding values of the one or more empirically-evaluated proteins to obtain at least a first principal component.

78. The method of embodiment 77, further comprising: generating, using the machine learning model, a training prediction of a molecular binding property of the one or more empirically-evaluated proteins; and comparing the training prediction and the first principal component to calculate the one or more cross-validation losses.

79. The method of embodiment 77 or 78, wherein the first principal component describes an average batch binding value.

80. The method of any one of embodiments 32-79, further comprising: generating, based on the prediction, a set of functions representing a behavior of the one or more proteins for a set of experimental conditions; and selecting at least one of the one or more proteins for one or more drug discovery assays based on the behavior of the one or more proteins for the set of experimental conditions.

81. The method of any one of embodiments 32-80, wherein the correlation between the selected representative feature vectors and the predetermined batch binding data associated with the one or more empirically-evaluated proteins comprises: a correlation between the representative feature vectors and a principal component calculated based on the predetermined batch binding data.

82. The method of embodiment 81, wherein the one or more cross-validation losses are calculated based on a predicted molecular binding property and an empirical molecular binding property.

83. The method of embodiment 82, wherein the predicted molecular binding property comprises a principal component calculated based on the representative feature vectors, and the empirical molecular binding property comprises a principal component calculated based on the predetermined batch binding data.

84. The method of any one of embodiments 32-83, wherein determining the one or more most-predictive feature vectors further comprises: (i) fitting a model to the representative feature vectors; (ii) calculating, based on the model, a feature importance score for each of the representative feature vectors; and (iii) removing one or more feature vectors of the representative feature vectors based on the feature importance score of each of the representative feature vectors to obtain a subset of representative feature vectors, wherein the one or more most-predictive feature vectors comprise one or more feature vectors from the subset of representative feature vectors.

85. The method of embodiment 84, further comprising: iteratively performing steps (i)-(iii) until a number of feature vectors included in the subset satisfies a feature quantity criterion.

86. The method of embodiment 85, wherein the feature quantity criterion being satisfied comprises the number of feature vectors included in the subset of representative feature vectors being less than or equal to a threshold number of feature vectors.

87. The method of embodiment 86, wherein the threshold number of feature vectors comprises a same or similar number of features from the training data used to train the machine learning model.

88. The method of any one of embodiments 84-87, wherein the number of feature vectors included in the subset of representative feature vectors comprises one of the set of hyper-parameters.

89. The method of any one of embodiments 32-88, wherein one of the set of hyper-parameters represents a number of feature vector clusters included in the plurality of feature vector clusters.

90. A system including one or more computing devices, the system further comprising: one or more non-transitory computer-readable storage media including instructions; and one or more processors coupled to the one or more storage media, the one or more processors configured to execute the instructions to effectuate the method of any one of embodiments 1-89.

91. A non-transitory computer-readable medium comprising instructions that, when executed by one or more processors of one or more computing devices, cause the one or more processors to effectuate operations comprising the method of any one of embodiments 1-89.

1. A method for predicting a molecular binding property of one or more proteins, comprising, by one or more computing devices:
accessing a molecular descriptor matrix representing a set of amino acid sequences corresponding to one or more proteins; and
refining a set of hyper-parameters associated with a machine learning model trained to generate a prediction of a molecular binding property of the one or more proteins, wherein refining the set of hyper-parameters comprises iteratively executing a process until a desired precision is reached, the process comprising:
reducing the molecular descriptor matrix by selecting one representative feature vector for each of a plurality of feature vector clusters, wherein each feature vector cluster includes similar feature vectors;
determining one or more most-predictive feature vectors of the selected representative feature vectors for each feature vector cluster based on a correlation between the selected representative feature vectors and predetermined batch binding data associated with the one or more proteins;
calculating one or more cross-validation losses based at least in part on the one or more most-predictive feature vectors and the predetermined batch binding data; and
updating the set of hyper-parameters based on the one or more cross-validation losses; and
outputting, by the machine learning model, the prediction of the molecular binding property of the one or more proteins based at least in part on the updated set of hyper-parameters.

2. The method of claim 1, wherein calculating the one or more cross-validation losses further comprises:
evaluating a cross-validation loss function based on the one or more most-predictive feature vectors, the predetermined batch binding data, the set of hyper-parameters, and a set of learnable parameters associated with the machine learning model; and
minimizing the cross-validation loss function by varying the set of learnable parameters while the one or more most-predictive feature vectors, the predetermined batch binding data, and the set of hyper-parameters remain constant.

3.-6. (canceled)

7. The method of claim 1, further comprising:
subsequent to refining the set of hyper-parameters:
accessing a second molecular descriptor matrix representing a second set of amino acid sequences corresponding to one or more second proteins;
reducing the second molecular descriptor matrix by selecting one representative feature vector for each of a second plurality of feature vector clusters of the second molecular descriptor matrix;
determining one or more second most-predictive feature vectors of the selected representative feature vectors for each feature vector cluster based on a second correlation between the selected representative feature vectors and predetermined batch binding data associated with the one or more second proteins;
inputting the one or more second most-predictive feature vectors into the machine learning model trained to generate a prediction of a molecular binding property of the one or more second proteins; and
outputting, by the machine learning model, the prediction of the molecular binding property of the one or more second proteins based at least in part on the updated set of hyper-parameters.

8. (canceled)

9. The method of claim 1, wherein the updated set of hyper-parameters comprises one or more of an updated set of general parameters, an updated set of booster parameters, or an updated set of learning-task parameters.

10. The method of claim 1, wherein calculating the one or more cross-validation losses comprises calculating an n number of cross-validation losses, and wherein n comprises an integer from 1-n.

11. The method of claim 1, wherein calculating the one or more cross-validation losses comprises determining an n number of individual train-test splits based on the one or more most-predictive feature vectors and the predetermined batch binding data, and wherein n comprises an integer from 1-n.

12. The method of claim 1, wherein calculating the one or more cross-validation losses comprises calculating an n number of cross-validation losses, the method further comprising:
generating the prediction of the molecular binding property of the one or more proteins based on an averaging of the n number of cross-validation losses.

13. The method of claim 1, wherein the molecular descriptor matrix comprises 2″ feature vectors, and wherein n comprises a dimension of the molecular descriptor matrix.

14. The method of claim 1, wherein the molecular descriptor matrix was generated by a first machine learning model distinct from the machine learning model.

15.-16. (canceled)

17. The method of claim 1, wherein the machine learning model comprises one or more of a gradient boosting model, an adaptive boosting (AdaBoost) model, an extreme gradient boosting (XGBoost) model, a light gradient boosted machine (LightGBM) model, or a categorical boosting (CatBoost) model.

18. The method of claim 1, wherein the machine learning model is further trained to generate a prediction of at least one of a molecular elution property or a flow-through property of the one or more proteins.

19. (canceled)

20. The method of claim 1, wherein reducing the molecular descriptor matrix comprises performing a Pearson's correlation of feature vectors of the molecular descriptor matrix to generate the plurality of feature vector clusters.

21. (canceled)

22. The method of claim 1, wherein determining the one or more representative feature vectors of the selected representative feature vectors for each of the plurality of feature vector clusters comprises selecting a k-best matrix of feature vectors of the selected representative feature vectors for each of the plurality of feature vector clusters.

23. (canceled)

24. The method of claim 1, wherein the correlation between the selected representative feature vectors and the predetermined batch binding data is determined based on a maximal information coefficient (MIC) between the selected representative feature vectors and the predetermined batch binding data.

25. The method of claim 1, wherein the prediction of the molecular binding property of the one or more proteins comprises a computational model-based chromatography process.

26.-28. (canceled)

29. The method of claim 1, wherein the prediction of the molecular binding property of the one or more proteins comprises an identification of a target protein of the one or more proteins.

30. The method of claim 1, wherein the prediction of the molecular binding property of the one or more proteins comprises a quantitative structure property relationship (QSPR) or a quantitative structure activity relationship (QSAR) modeling of the one or more proteins.

31. The method of claim 1, wherein the prediction of the molecular binding property of the one or more proteins comprises a prediction of a molecular binding property for each amino acid sequence of the set of amino acid sequences corresponding to the one or more proteins.

32. A system including one or more computing devices, the system further comprising:
one or more non-transitory computer-readable storage media including instructions; and
one or more processors coupled to the one or more storage media, the one or more processors configured to execute the instructions to:
access a molecular descriptor matrix representing a set of amino acid sequences corresponding to one or more proteins; and
refine a set of hyper-parameters associated with a machine learning model trained to generate a prediction of a molecular binding property of the one or more proteins, wherein refining the set of hyper-parameters comprises iteratively executing a process until a desired precision is reached, the process comprising instructions to:
reduce the molecular descriptor matrix by selecting one representative feature vector for each of a plurality of feature vector clusters, wherein each of the feature vector clusters includes similar feature vectors;
determine one or more representative feature vectors of the selected representative feature vectors for each of the plurality of feature vector clusters based on a correlation between the selected representative feature vectors and predetermined batch binding data associated with the one or more proteins;
calculate one or more cross-validation losses based at least in part on the one or more most-predictive feature vectors and the predetermined batch binding data; and
update the set of hyper-parameters based on the one or more cross-validation losses; and
output, by the machine learning model, the prediction of the molecular binding property of the one or more proteins based at least in part on the updated set of hyper-parameters.

33.-62. (canceled)

63. A non-transitory computer-readable medium comprising instructions that, when executed by one or more processors of one or more computing devices, cause the one or more processors to:
access a molecular descriptor matrix representing a set of amino acid sequences corresponding to one or more proteins; and
refine a set of hyper-parameters associated with a machine learning model trained to generate a prediction of a molecular binding property of the one or more proteins, wherein refining the set of hyper-parameters comprises iteratively executing a process until a desired precision is reached, the process comprising instructions to:
reduce the molecular descriptor matrix by selecting one representative feature vector for each of a plurality of feature vector clusters, wherein each of the feature vector clusters includes similar feature vectors;
determine one or more representative feature vectors of the selected representative feature vectors for each of the plurality of feature vector clusters based on a correlation between the selected representative feature vectors and predetermined batch binding data associated with the one or more proteins;
calculate one or more cross-validation losses based at least in part on the one or more most-predictive feature vectors and the predetermined batch binding data; and
update the set of hyper-parameters based on the one or more cross-validation losses; and
output, by the machine learning model, the prediction of the molecular binding property of the one or more proteins based at least in part on the updated set of hyper-parameters.

64.-153. (canceled)
